(19) United States
(12) Patent Application Publication
(10) Pub. No.: US 2025/0380962 A1
(43) Pub. Date: Dec. 18, 2025

(54) TISSUE CUTTING TOOL

(71) Applicant: EDWARDS LIFESCIENCES CORPORATION, Irvine, CA (US)

(72) Inventors: Peleg Harel, Haifa (IL); Ofir Witzman, Harish (IL); Eitan Atias, Tel Aviv (IL)

(73) Assignee: EDWARDS LIFESCIENCES CORPORATION, Irvine, CA (US)

(21) Appl. No.: 19/302,513

(22) Filed: Aug. 18, 2025

Related U.S. Application Data
(63) Continuation of application No. PCT/US2024/017639, filed on Feb. 28, 2024.
(60) Provisional application No. 63/449,557, filed on Mar. 2, 2023.
(60) Provisional application No. 63/556,466, filed on Feb. 22, 2024.

(51) Int. Cl. A61B 17/3205; A61B 17/34; A61M 29/00

(52) U.S. Cl. CPC A61B 17/32053; A61B 17/34; A61M 29/00; A61B 2560/04

(57) ABSTRACT

The present disclosure relates to tissue cutting tools that can be used for forming an opening in a target tissue, such as a host leaflet within which a guest prosthetic valve can be expanded. In an example, the tissue cutting tool comprises an actuation tube and a blade support tube extending therethrough. A distal portion of the blade support tube can be coupled to a distal portion of the actuation tube, and a proximal portion of the blade support tube can be axially movable with respect to a proximal portion of the actuation tube. The blade support tube also includes a bendable section extending between its distal and proximal portions, and configured to transition between unbent and bent configuration thereof. At least one blade is coupled to at least one arm of the bendable section.

### CROSS-REFERENCE TO RELATED APPLICATIONS

This application is a continuation of International Application No. PCT/US2024/017639, filed Feb. 28, 2024, which claims the benefit of U.S. Provisional Application No. 63/449,557, filed Mar. 2, 2023, and U.S. Provisional Application No. 63/556,466, filed Feb. 22, 2024, the contents of which are herein incorporated by reference in their entirety.

### FIELD

The present disclosure relates to tissue cutting tools that can be used to form an opening in a target tissue, and to methods and devices for cutting through a target tissue that can be a leaflet of an existing valvular structure, in a manner that can modify existing valvular structures (for example, leaflets of a native heart valve or previously-implanted prosthetic valve) prior to implantation of a guest prosthetic valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), or transcatheter mitral valve replacement (TMVR), are used in various procedures to deliver prosthetic medical devices to locations inside the body that are not readily accessible by surgery or where access without surgery is desirable.

Ischemic heart disease can lead to valve regurgitation, such as mitral regurgitation. This is caused by the combination of weakened papillary muscles and dilation of the left ventricle, which displaces the papillary muscles and enlarges the annulus of the mitral valve. This prevents the leaflets from sealing properly when the valve is closed, resulting in blood flowing back from the left ventricle into the left atrium-a condition known as mitral regurgitation. This increases total stroke volume and decreases cardiac output, leading to the weakening of the left ventricle due to volume and pressure overload of the left atrium. Transcatheter mitral valve replacement (TMVR) is one example of a minimally-invasive surgical procedure used to replace a native mitral valve.

Transcatheter aortic valve replacement (TAVR) is another example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

One known technique for mitigating the risk of coronary ostial obstruction involves lacerating or severing a portion of one or more leaflets of the host valve (which can be an aortic bioprosthetic valve or a native aortic valve). Lacerating or severing a portion of the leaflet(s) reduces the risk of blocking the coronary ostia when the guest prosthetic valve is implanted and displaces the leaflets of the host valve toward the inner wall of the aortic root. However, methods that rely on lacerating existing leaflets, require high spatial precision and surgical skill. Moreover, once the leaflets have been lacerated, the existing heart valve may function poorly and increase the risk of aortic insufficiency, at least until a replacement prosthetic valve has been successfully implanted. If the existing leaflets have become calcified, there is a further risk that the lacerating will release particulate or other debris into the blood stream, which may make the patient susceptible to vascular occlusion or stroke.

In the case of mitral regurgitation, prosthetic valves can be delivered to a malfunctioning native mitral valve, and expand against the native annulus and leaflets. In some cases, expansion of the prosthetic valve pushes the anterior leaflet of the mitral valve into the left ventricular outflow tract (LVOT), which may significantly limit outflow through the LVOT and the aortic valve.

In one of its basic configurations, a tissue cutting tool comprises an actuation tube and a blade support tube. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the actuation tube can comprise an actuation tube distal portion and an actuation tube proximal portion.

In some examples, the blade support tube can comprise a support tube distal portion attached to the actuation tube distal portion.

In some examples, the blade support tube can comprise a support tube proximal portion extending through the actuation tube proximal portion.

In some examples, the blade support tube can comprise a bendable section extending between the support tube distal portion and the support tube proximal portion.

In some examples, the blade support tube can comprise at least one blade coupled to at least one arm of the bendable section.

In some examples, axial movement of the actuation tube proximal portion and/or the support tube proximal portion relative to each other is optionally configured to transition the bendable section between unbent and bent configurations thereof.

In some examples, the at least one blade is optionally oriented at an angle relative to the actuation tube when the bendable section is in the bent configuration.

In some examples, the at least one arm can comprise a proximal arm and a distal arm.

In some examples, the at least one blade can comprise a blade attached to the proximal arm.

In some examples, the tissue cutting tool can comprise a gathering arm attached, at a gathering arm distal portion thereof, to the distal arm.

In some examples, the gathering arm can comprise a gathering arm proximal portion extending proximally from the gathering arm distal portion.

In some examples, the gathering arm proximal portion is optionally oriented radially away from the blade in the bent configuration of the bendable section.

In some examples, the at least one blade can comprise a blade attached to the distal arm.

In some examples, the actuation tube can comprise a cutout extending between the actuation tube distal portion and the actuation tube proximal portion.

In some examples, the bendable section can optionally protrude radially away from the cutout in the bent configuration.

In some examples, the actuation tube can comprise an actuation tube distal edge configured to perforate a target tissue.

In some examples, the tissue cutting tool can comprise a needle extending through the blade support tube.

In one of its basic methods, a method of forming an opening in a target tissue comprises advancing a tissue cutting tool to a target tissue. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the tissue cutting tool optionally comprises an actuation tube and a blade support tube.

In some examples, the actuation tube optionally comprises an actuation tube distal portion and an actuation tube proximal portion.

In some examples, the blade support tube optionally comprises a support tube proximal portion extending through the actuation tube proximal portion.

In some examples, the blade support tube optionally comprises a support tube distal portion attached to the actuation tube distal portion.

In some examples, the blade support tube optionally comprises at least one blade coupled to at least one arm of a bendable section of the blade support tube.

In some examples, the bendable section of the blade support tube can optionally extend between the support tube distal portion and the support tube proximal portion.

In some examples, the method comprises transitioning the bendable section from an unbent configuration thereof to a bent configuration.

In some examples, the method comprises forming a tissue opening within the target tissue by axially moving the bendable section through the target tissue.

In some examples, the actuation tube optionally comprises a cutout extending between the actuation tube distal portion and the actuation tube proximal portion.

In some examples, the transitioning the bendable section to the bent configuration can optionally cause the bendable section to protrude radially away from the cutout.

In some examples, the method optionally comprises, prior to the forming the tissue opening, forming a pilot puncture within the target tissue.

In some examples, the forming the tissue opening optionally comprises passing the bendable section through the pilot puncture, such that the at least one blade cuts through the target tissue so as to expand the pilot puncture to form the tissue opening.

In some examples, the tissue cutting tool optionally comprises a needle extending through the blade support tube.

In some examples, the forming the pilot puncture optionally comprises distally translating the needle through the target tissue.

In some examples, the tissue cutting tool optionally comprises a dilator attached to the actuation tube, wherein the dilator optionally comprises a dilator distal tapering surface that tapers towards a distal end of the dilator.

In some examples, the method optionally comprises, subsequent to the forming the pilot puncture and prior to the forming the tissue opening, passing the dilator through the pilot puncture, thereby expanding the pilot puncture.

In some examples, the actuation tube optionally comprises an actuation tube distal edge configured to cut through the target tissue.

In some examples, the forming the pilot puncture optionally comprises distally translating the actuation tube distal edge through the target tissue.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a cross-sectional view of a native aortic valve.

FIG. 2A shows a cross-sectional view of a prosthetic heart valve implanted in the native aortic valve of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4A is a side view of an exemplary tissue cutting tool, shown in an unbent configuration of a bendable section thereof.

FIG. 4B is a side view of the tissue cutting tool of FIG. 4A, shown in a bent configuration of the bendable section.

FIG. 5A is a cross-sectional view of the tissue cutting tool of FIG. 4A, shown in the unbent configuration.

FIG. 5B is a cross-sectional view of the tissue cutting tool of FIG. 4B, shown in the bent configuration.

FIG. 6 shows an exemplary retention portion of an arm of the bendable section.

FIG. 7 shows an exploded view of an exemplary blade prior to being coupled to the retention portion of FIG. 6.

FIG. 8 shows the blade and retention portion of FIG. 7 attached to each other.

FIG. 9 shows a distal portion of an exemplary blade support tube.

FIG. 10 shows the distal portion of the blade support tube of FIG. 9 engaged with a distal portion of an exemplary actuation tube.

FIG. 11A is a simplified side view of a tissue cutting tool positioned on an outflow side of a host leaflet according to an example.

FIG. 11B is a simplified side view of the tissue cutting tool of FIG. 11A with an actuation tube piercing the host leaflet.

FIG. 11C is a simplified side view of the tissue cutting tool of FIG. 11A with a bendable exposed in an unbent configuration distal to the host leaflet.

FIG. 11D is a simplified side view of the tissue cutting tool of FIG. 11A with the bendable section transitioned to the bent configuration, distal to the host leaflet.

FIG. 11E is a simplified side view of the tissue cutting tool of FIG. 11A with a blade coupled to the bendable section cutting through the host leaflet as it is pulled therethrough.

FIG. 11F is a simplified side view of the tissue cutting tool of FIG. 11A with the bendable section transitioned to the folded configuration subsequent to formation of a leaflet opening in the host leaflet.

FIG. 11G is a simplified side view of the tissue cutting tool of FIG. 11A with the bendable section covered by a cover shaft.

FIG. 12 shows a previously implanted prosthetic valve subsequent to forming the leaflet opening in a host leaflet thereof.

FIG. 13 shows a configuration in which a guest prosthetic valve has been expanded within the leaflet opening of a host prosthetic valve.

FIG. 14A is a simplified side view of a tissue cutting tool positioned on an outflow side of a host leaflet according to an example.

FIG. 14B is a simplified side view of the tissue cutting tool of FIG. 14A with the bendable exposed in an unbent configuration proximal to the host leaflet.

FIG. 14C is a simplified side view of the tissue cutting tool of FIG. 14A with the bendable section transitioned to the bent configuration, proximal to the host leaflet.

FIG. 14D is a simplified side view of the tissue cutting tool of FIG. 14A with a blade coupled to the bendable section cutting through the host leaflet as it is pushed therethrough.

FIG. 15A is a cross-sectional view the right-side of a human heart.

FIG. 15B shows an exemplary prosthetic valve implanted in the native mitral valve.

FIG. 16A is a side view of an exemplary tissue cutting tool that includes a gathering arm, shown in an unbent configuration of a bendable section thereof.

FIG. 16B is a side view of the tissue cutting tool of FIG. 16A, shown in a bent configuration of the bendable section.

FIG. 17A is a cross-sectional view of the tissue cutting tool of FIG. 16A, shown in the unbent configuration.

FIG. 17B is a cross-sectional view of the tissue cutting tool of FIG. 16B, shown in the bent configuration.

FIG. 18 is a perspective view of a distal portion of the tissue cutting tool of FIG. 16B.

FIG. 19A is a perspective view of a distal portion of the tissue cutting tool of FIGS. 16A-18, having a dilator shown with transparency for illustrative purpose.

FIG. 19B is a cross-sectional side view of the portion of the tissue cutting tool shown in FIG. 19A.

FIG. 20 shows a tissue cutting tool advanced transseptally to the left atrium.

FIGS. 21A-21F illustrate steps in an exemplary method for utilizing the tissue cutting tool for cut through a host leaflet.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient's heart. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

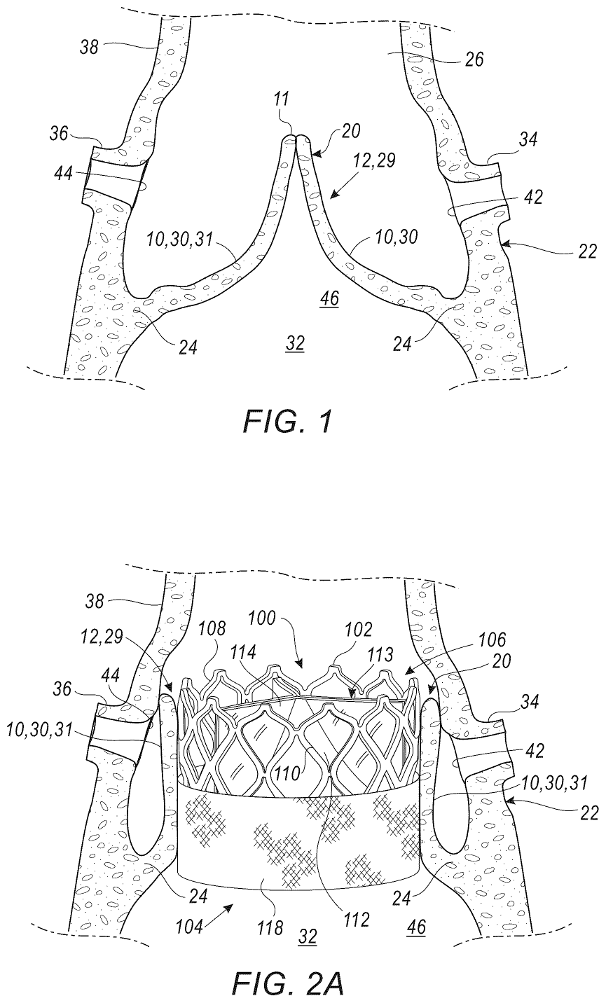

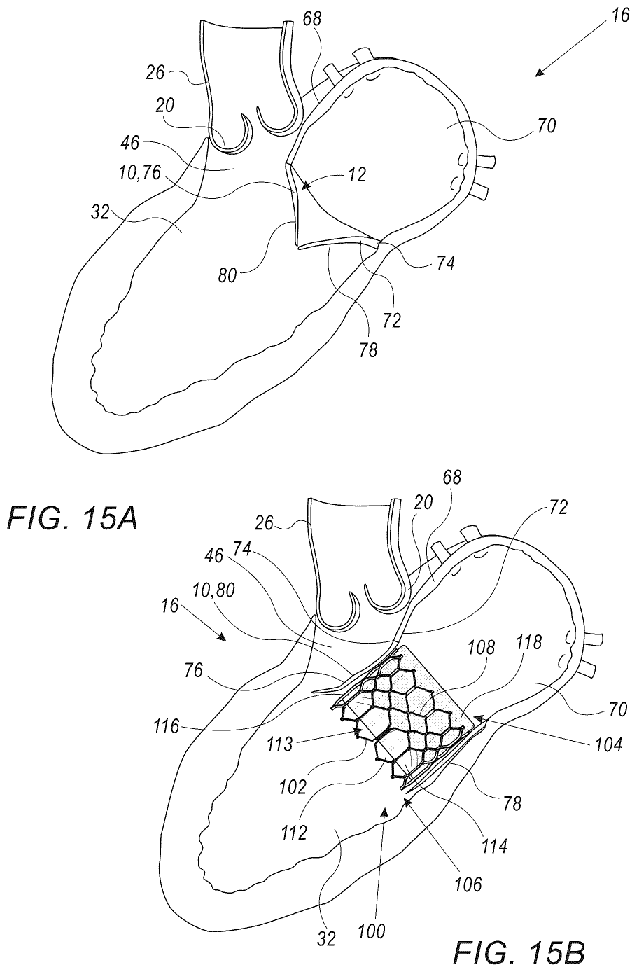

FIG. 1 illustrates an anatomy of the aortic root 22, which is positioned between the left ventricle 32 and the ascending aorta 26. A native heart valve generally includes a native valvular structure 29 comprising a plurality of native leaflets 30, which can be, for example, aortic leaflets 31 in the case of a native aortic valve 20 illustrated in FIG. 1, or mitral leaflets 76 in the case of a native mitral valve 72 illustrated in FIG. 15A. The aortic root 22 includes a native aortic valve 20 having a native valvular structure 29 comprising a plurality of native aortic leaflets 31. Normally, the native aortic valve 20 has three aortic leaflets 31 (only two leaflets are visible in the simplified illustration of FIG. 1), but aortic valves with fewer than three leaflets are possible. The leaflets 31 are supported at native commissures 40 (see FIG. 1B) by the aortic annulus 24, which is a ring of fibrous tissue at the transition point between the left ventricle 32 and the aortic root 22, and terminate at free edges 11 opposite to the commissures. The leaflets 30, 31 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

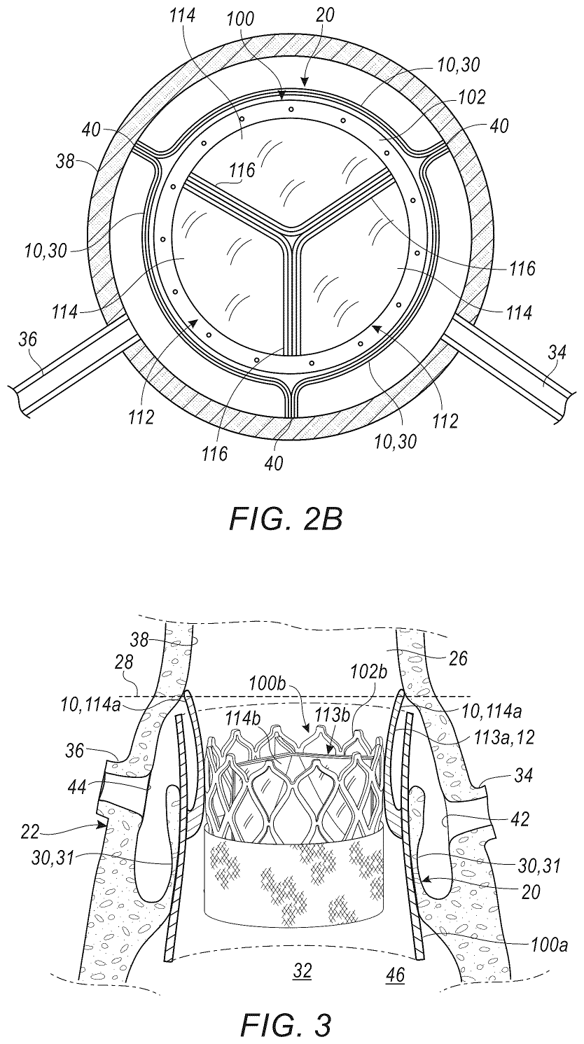

FIGS. 2A-2B show an exemplary prosthetic valve 100 that can optionally be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can optionally be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/85,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can optionally be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can optionally be made of various suitable materials, including plastically-deformable materials such as, but not limited to, stainless steel, a nickel-based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can optionally be made of shape-memory materials such as, but not limited to, nickel-titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 can optionally be an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can optionally include a plurality of angled struts and vertical or axial struts. At least some of the struts 108 can be pivotable or bendable relative to each other, so as to permit frame expansion or compression. For example, the frame 102 can optionally be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can optionally include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can optionally be arranged together to form prosthetic valve commissures 116 that are coupled (directly or indirectly) to respective portions of the frame 102, thereby securing at least a portion of the valvular structure 113 to the frame 102. The prosthetic valve leaflets 114 can optionally be made from, in whole or part, biological material (for example, pericardium), biocompatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can optionally comprise at least one skirt or sealing member. For example, the prosthetic valve 100 can optionally include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can optionally be sutured to the inner skirt along a scalloped line (not shown). An inner skirt can optionally be coupled to the frame 102 via sutures or another form of coupler.

The prosthetic valve 100 can optionally comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can optionally be formed of a single sheet of material that extends continuously around the inner surface of frame 102. In some cases, the outer skirt 118 can optionally be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell openings 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. Positioning of the prosthetic valve 100 can be such that an inflow portion thereof extends into the left ventricle outflow tract (LVOT) 46. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can optionally be a surgically implanted prosthetic heart valve (known as a “surgical valve”) or a transcatheter heart valve. The term “guest valve”, as used herein, refers to a prosthetic valve implanted in a host valve, which can optionally be either a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36. Any delivery apparatus described throughout the current disclosure, is advantageously configured to modify the host valvular structure 12 (i.e., modify at least one of the host leaflets 10), and optionally implant a guest prosthetic valve 100 within the modified valvular structure 12, optionally without the need to switch between separate delivery apparatuses for each function.

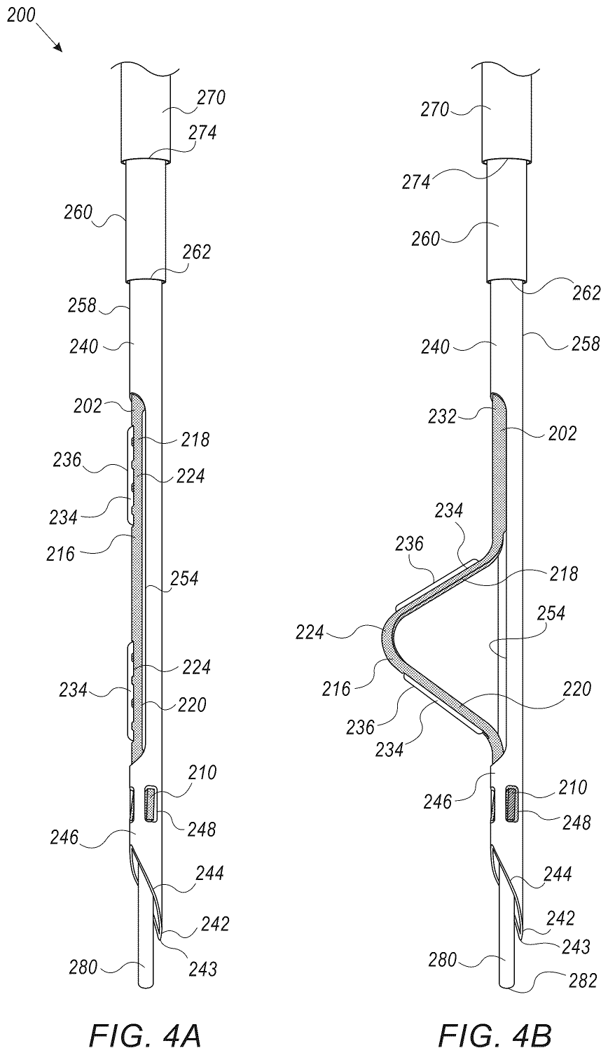

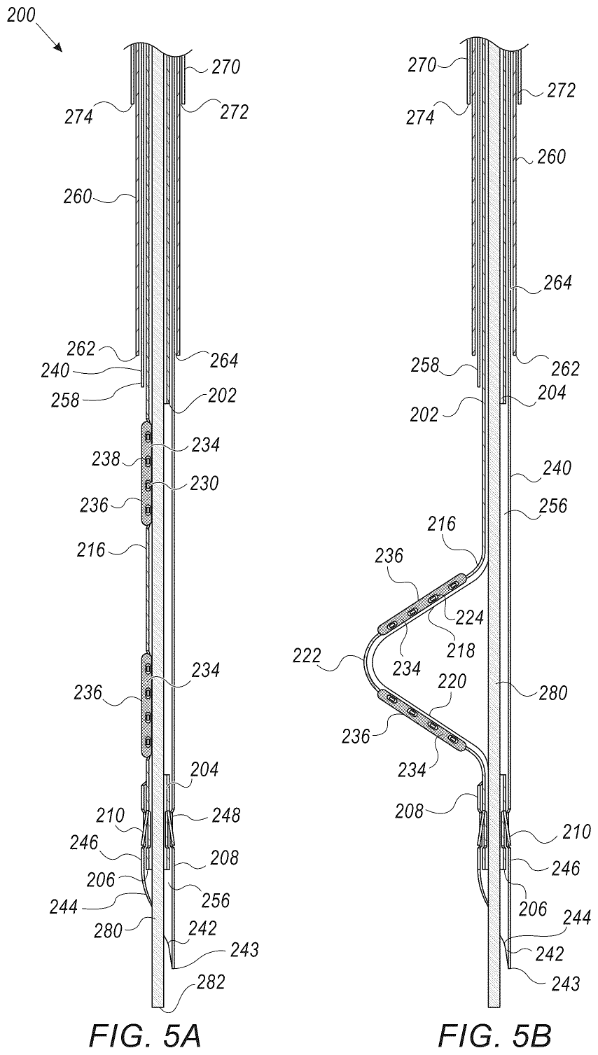

FIGS. 4A-5B show an exemplary tissue cutting tool 200 that can optionally be used to perforate and/or cut a tissue, such as a host leaflet 10 of a host valvular structure 12. The tissue cutting tool 200 comprises a blade support tube 202 having a bendable section 216 to which at least one blade 234 is attached, wherein the bendable section 216 is configured to transition between bent and unbent configurations. FIGS. 4A and 4B show perspective side views of a distal portion of the tissue cutting tool 200 in unbent and bent configurations, respectively, of its bendable section 216. FIGS. 5A and 5B show cross-sectional side views of the tissue cutting tool 200 in the configurations illustrated in FIGS. 4A and 4B, respectively.

The blade support tube 202 can optionally define a support tube lumen 204, and optionally include a support tube distal portion 208 extending distally to the bendable section 216, and a support tube proximal portion 232 extending proximally from the bendable section 216. The support tube distal portion 208 and support tube proximal portion 232 can optionally have a circular or tubular cross-section, while the bendable section 216 does not enclose a full circular perimeter in cross-section, but can be rather formed with a cutout such that the cross-sectional shape of the bendable section 216 forms an arc to allow it to easily transition to and from a bent configuration. The support tube lumen 204 can optionally be sized to allow axial passage of a guidewire 280 therethrough.

The tissue cutting tool 200 can optionally further comprise an actuation tube 240 disposed around the blade support tube 202. The actuation tube 240 defines an actuation tube lumen 256, and includes an actuation tube distal portion 246 optionally coupled to the support tube distal portion 208. The actuation tube distal portion 246 comprises an actuation tube distal edge 242 that can optionally be configured to pierce a host leaflet 10 of a host valvular structure 12 to form a pilot puncture 50 in the host leaflet 10. The actuation tube distal edge 242 can optionally extend along an angled surface 244 that can optionally have a sharp actuation tube tip 243 to facilitate piercing the host leaflet 10 when the needle is pressed against the leaflet.

The actuation tube 240 can optionally further comprise an actuation tube proximal portion 258 disposed around the support tube proximal portion 232, and a cutout 254 extending between the actuation tube distal portion 246 and the actuation tube proximal portion 258. The cutout 254 can optionally be aligned with the bendable sections 216 of the blade support tube 202, and is configured to expose the bendable section 216 and allow it to protrude radially outward therethrough in the bent configuration, while the actuation tube distal portion 246 extends around the support tube distal portion 208 and the actuation tube proximal portion 258 is disposed around the support tube proximal portion 232. Stated differently, the arms 218, 220, as well as any blade 234 attached thereto, are oriented at an angle with respect to the actuation tube 240 in the bent configuration.

The actuation tube lumen 256 can optionally be sized to accommodate the blade support tube 202 therein, and specifically to accommodate the support tube distal portion 208 within the actuation tube distal portion 246 and the support tube proximal portion 232 within the actuation tube proximal portion 258.

The tissue cutting tool 200 can optionally further comprise a cover shaft 260 disposed around the actuation tube 240. The cover shaft 260 defines a cover shaft lumen 264 and terminates at a cover shaft distal edge 262. The actuation tube 240 can optionally be axially movable relative to the cover shaft 260. The cover shaft lumen 264 can optionally be sized to allow passage of the actuation tube 240 and blade support tube 202 therethrough.

The tissue cutting tool 200 can optionally further comprise a catheter 270 disposed around the cover shaft 260. The catheter 270 defines a catheter lumen 272 and terminates at a catheter distal edge 274. The cover shaft 260 and actuation tube 240 can optionally be axially movable relative to catheter 270. The catheter lumen 272 can optionally be sized to allow passage of the cover shaft 260 therethrough.

The support tube distal portion 208 terminates at a support tube distal edge 206, which can optionally be proximal to the actuation tube distal edge 242. The support tube distal portion 208 can optionally be attached to the actuation tube distal portion 246 in a manner that prevents axial movement therebetween, while the support tube proximal portion 232 and the actuation tube proximal portion 258 can optionally be axially movable relative to each other. Thus, in such cases, axial movement between the support tube proximal portion 232 and the actuation tube proximal portion 258, while the support tube distal portion 208 remains immovable with respect to the actuation tube distal portion 246, serves to transition the bendable section 216 between the unbent and bent configuration. For example, axially pulling the actuation tube 240 relative to the blade support tube 202, and/or axially pushing the blade support tube 202 relative to the actuation tube 240, serves to transition the bendable section 216 from the unbent configuration of FIGS. 4A and 5A, to the bent configuration shown in FIGS. 4B and 5B. Similarly, axially pulling the blade support tube 202 relative to the actuation tube 240, and/or axially pushing the blade support tube 202 relative to the actuation tube 240, serves to transition the bendable section 216 from the bent configuration of FIGS. 4B and 5B back to the unbent configuration of FIGS. 4A and 5A.

In the bent configuration, the bendable section 216 extends radially away from the longitudinal axis of the tissue cutting tool 200, forming an intermediate bend 222 offset radially farther away from the cutout 254 relative to the unbent configuration. The bendable section 216 includes a proximal arm 218 extending between the support tube proximal portion 232 and the intermediate bend 222, and a distal arm 220 extending between the intermediate bend 222 and the support tube distal portion 208. At least one of the arms 218, 220 can optionally further include a blade 234 attached thereto, the blade 234 having a sharp edge 236 facing radially away from the respective arm 218, 220.

In some examples, each of the proximal arms 218 and distal arm 220 can optionally comprise a blade 234 attached thereto, as shown in the illustrated example. However, it is to be understood that in some examples, only one of the arms can be equipped with a blade 234, while the other arm can be devoid of a blade. For example, the proximal arms 218 can include a blade 234 attached thereto, while the distal arm 220 is devoid of a blade, or the distal arm 220 can include a blade 234 attached thereto, while the proximal arm 218 is devoid of a blade (examples not illustrated explicitly).

While a single blade 234 is attached to each arm 218, 220 in the illustrated example, it is to be understood that any of the proximal arm 218 and/or distal arm 220 can optionally include more than one blade 234 attached thereto.

In the unbent configuration, as shown in FIGS. 4A and 5A, the arms 218, 220 and the blades 234 are substantially parallel to the longitudinal axis of the tissue cutting tool 200, while in the bent configuration, as shown in FIGS. 4B and 5B, the arms 218, 220 and the blades 234 are angled relative to the longitudinal axis of the tissue cutting tool 200 and relative to the remainder of the blade support tube 202 and/or actuation tube 240, such that the sharp edge 236 of a blade 234 coupled to the distal arm 220 is oriented in a distal direction, while the sharp edge 236 of a blade 234 coupled to the proximal arm 218 is oriented in a proximal direction.

A blade 234 can optionally be coupled to any of the arms 218, 220 by any suitable means, such as by gluing, welding, and the like. The blade support tube 202, or at least the bendable section 216 thereof, can optionally be made from a flexible material that can transition between bent and unbent configurations. In some examples, the blade support tube 202, or at least the bendable section 216 thereof, can optionally be made of a shape memory material, such as Nitinol. While many metallic components can be attached to each other by welding, Nitinol is not easily welded to other components, and may therefor require alternative dedicated mechanical attachment configurations.

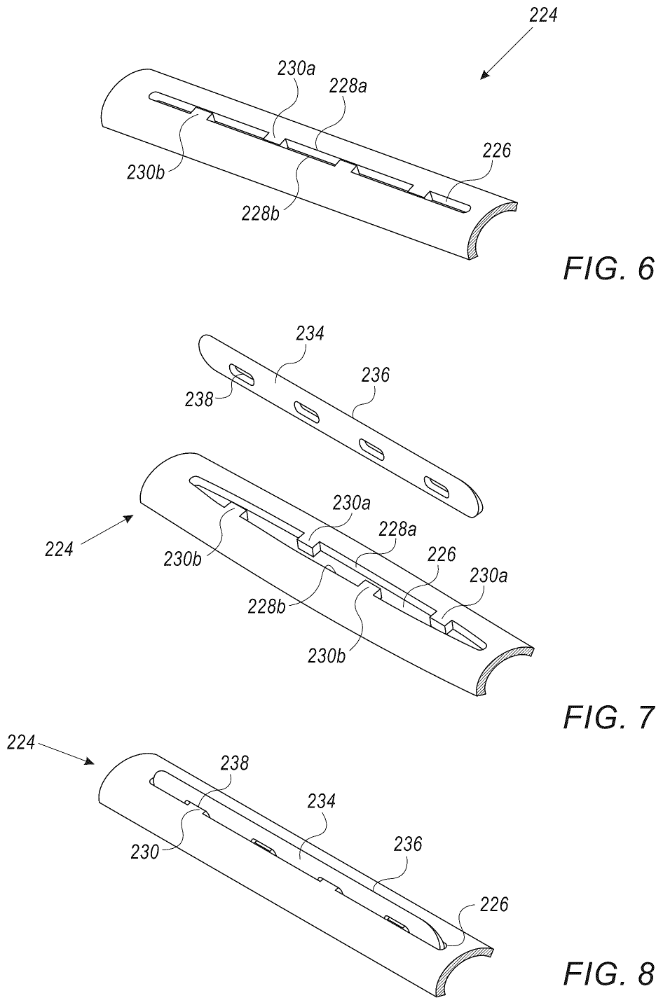

FIGS. 6-8 illustrate an exemplary manner by which a blade 234 can optionally be coupled to any of the arms 218, 220 of the blade support tube 202. Any of the proximal arm 218 and/or distal arm 220 can include a retention portion 224 to which a blade 234 can optionally be coupled. FIG. 6 shows an exemplary retention portion 224 that can optionally include a longitudinal slot 226 extending through the thickness of the bendable section's wall, defining two slot sidewalls 228 extending longitudinally and facing each other, and optionally formed so as to include one or more protrusions 230 extending from one sidewall toward the opposite sidewall. For example, a plurality of first protrusions 230a can optionally extend from a first slot sidewall 228a towards the second slot sidewall 228b, and a plurality of second protrusions 230b can optionally extend from the second slot sidewall 228b towards the first slot sidewall 228a. Each protrusion 230 can optionally be integrally formed with the sidewall it extends from, and terminate at a free edge which is not attached to the opposite sidewall.

While a total of four protrusions 230 is illustrated in FIG. 6, including two first protrusions 230a and two second protrusions 230b, arranged in an alternating manner, it is to be understood that any other number of protrusions 230 and arrangements thereof is contemplated. For example, any slot sidewall 228 can optionally include a single protrusion 230 or more than two protrusions 230, and any slot sidewall 228 can optionally include a different number of protrusions that the opposite slot sidewall 228. In some examples, one slot sidewall 228 can optionally include one or more protrusion 230, while the opposite slot sidewall 228 can optionally be devoid of protrusion. Likewise, the protrusions 230 can be equally spaced from each other and/or from opposing protrusions 230, or unequally spaced from each other.

FIG. 7 shows an exploded view of the blade 234 and the retention portion 224 separated from each other. As shown, the blade 234 can optionally include one or more side opening 238, optionally matching in number to the number of protrusions 230, and sized to allow extension of protrusions 230 therethrough. The position and spacing between adjacent side openings 238 can optionally match the position and distance between adjacent corresponding protrusions 230.

Nitinol, from which the retention portion 224 can optionally be made, exhibits the shape memory effect and superelasticity at different temperatures. As a result, the retention portion 224 may be deformed when cold, while returning to its pre-deformed (“memorized”) shape when heated. More specifically, below the Nitinol transformation temperature the retention portion 224 transforms to a martensite structure, which exhibits superelasticity, and thus its slot 226 can be expanded (due to the circumferentially applied pull forces from both sides of the slot) as shown in FIG. 7, distancing the slot sidewalls 228 and the protrusions 230 extending therefrom farther away from the opposite slot sidewalls 228, creating a gap large enough to allow positioning of the blade 234 in the slot 226 with the side openings 238 aligned with the protrusions 230. When the temperature is above the Nitinol transformation temperature (wherein the room temperature and/or body temperature can be above the transformation temperature), the retention portion 224 assumes an austenite structure and spontaneously reverts back to its non-expanded conformation shown in FIG. 6, closing the protrusions 230 inside the side openings 238, thereby affixing the blade 234 to the retention portion 224 as shown in FIG. 8.

In some examples, expanding the slot 226 so as to position the blade 234 therein can optionally be performed by application of mechanical forces on both sides of the slot 226 by a dedicated jig or other mechanical expansion device (not shown), in addition to, or instead of, relying on changing the temperature below and above a transformation temperature.

The support tube distal portion 208 can optionally be coupled to the actuation tube distal portion 246 by any suitable means, such as by gluing, welding, and the like. In some cases, the support tube distal portion 208 can optionally be made of Nitinol, which may be difficult to attach by welding to other tubes from the same reasons mentions above, and thus require alternative dedicated mechanical attachment configurations.

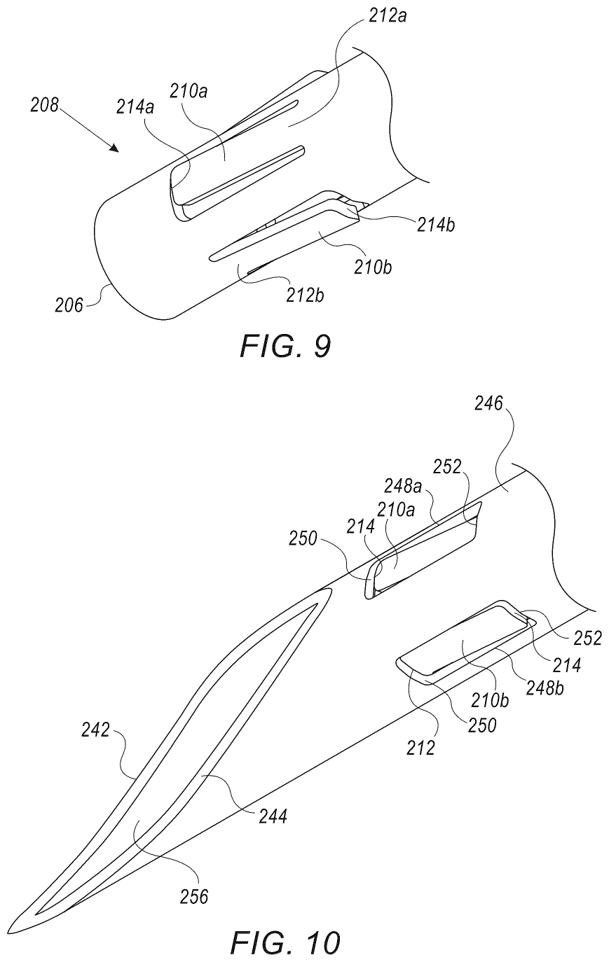

FIGS. 9-10 illustrate an exemplary manner by which support tube distal portion 208 can b optionally e coupled to the actuation tube distal portion 246. FIG. 9 illustrates an exemplary support tube distal portion 208 formed to include a plurality of tabs 210 proximal to the support tube distal edge 206. The tabs 210 can optionally be arranged around the circumference of support tube distal portion 208, wherein each tab 210 extends from a tab base 212 to a tab free end 214. Each tab 210 can be optionally formed by cutting a U-shaped slot, wherein the uncut end forms the tab base 212 which can optionally be integrally formed with the support tube distal portion 208.

The plurality of tabs 210 can optionally include at least two oppositely oriented tabs 210, comprising at least one distally oriented tab 210a with a distal tab free end 214a formed at its distal edge, and at least one proximally oriented tab 210b with a proximal tab free end 214b. While a total of four tabs 210 is illustrated in FIG. 9, including two distally oriented tabs 210a with distal tab free ends 214a and two proximally oriented tabs 210b with proximal tab free ends 214b, arranged in an alternating manner, it is to be understood that any other number of tabs 210 and arrangements thereof is contemplated. For example, support tube distal portion 208 can optionally include a single distally oriented tab 210a or more than two or more than two distally oriented tabs 210a, one proximally oriented tab 210b or more than two proximally oriented tabs 210b. The number of distally oriented tabs 210a can be similar to or different than the number of proximally oriented tabs 210b, and the tabs 210 can be equally spaced from each other or unequally spaced from each other in the circumferential direction.

FIG. 10 shows an exemplary actuation tube distal portion 246 with the support tube distal portion 208 coupled thereto. The actuation tube distal portion 246 can optionally include a plurality of windows 248 matching in number the plurality of tabs 210, circumferentially arranged around the circumference of the actuation tube distal portion 246, positioned and sized to accommodate the tabs 210. Each window 248 extends between a window distal edge 250 and a window proximal edge 252. The tabs 210 can optionally be biased radially outward, extending radially away from the tab base 212 to the tab free end 214, such that the tab free end 214 is disposed at the level of the edges 250, 252 of the windows 248.

When the tabs 210 are cut from the support tube distal portion 208, they can optionally be substantially flush with the rest of the outer surface of the support tube distal portion 208. The tabs can optionally be shape-set, when formed of a shape memory material such as Nitinol, to assume the illustrated angled configuration biased radially outwards. The support tube distal portion 208 can be cooled to a temperature below the Nitinol transformation temperature to transform the support tube distal portion 208 to the martensite structure, during which the tabs 210 can optionally be pressed so as to assume a flush configuration with the remainder of the support tube distal portion 208, allowing insertion thereof into actuation tube lumen 256, aligning the tabs 210 with corresponding windows 248 of the actuation tube distal portion 246. When the temperature is above the Nitinol transformation temperature, the tabs 210 can optionally assume their pre-shaped angled configuration, such that the tab free ends 214 are aligned with edges of the windows 248.

As shown in FIG. 10, a distal tab free end 214a of each distally oriented tab 210a is facing a window distal edge 250, while a proximal tab free end 214b of each proximally oriented tab 210b is facing a window proximal edge 252. The distal tab free ends 214a, abutting window distal edges 250, prevent a distally oriented movement of the support tube distal portion 208 relative to the actuation tube distal portion 246, and/or a proximally oriented movement of the actuation tube distal portion 246 relative to the support tube distal portion 208. Similarly, the proximal tab free ends 214b, abutting window proximal edges 252, prevent a proximally oriented movement of the support tube distal portion 208 relative to the actuation tube distal portion 246, and/or a distally oriented movement of the actuation tube distal portion 246 relative to the support tube distal portion 208. Thus, the inclusion of both distally oriented tabs 210a and proximally oriented tabs 210b can advantageously prevent axial movement between the support tube distal portion 208 and the actuation tube distal portion 246 both in the distal and proximal directions.

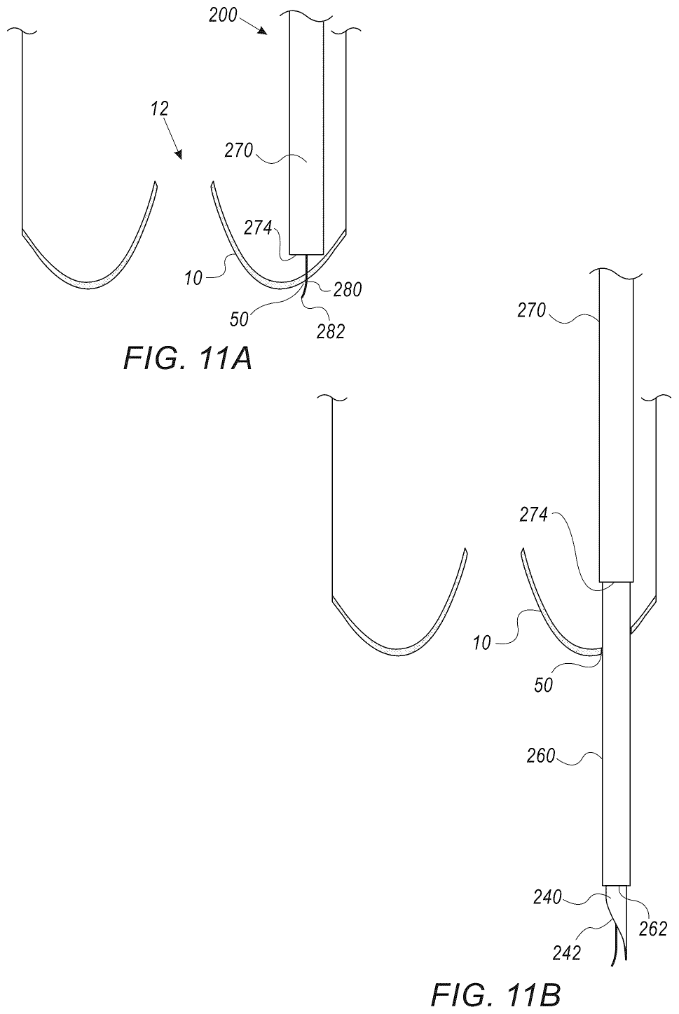

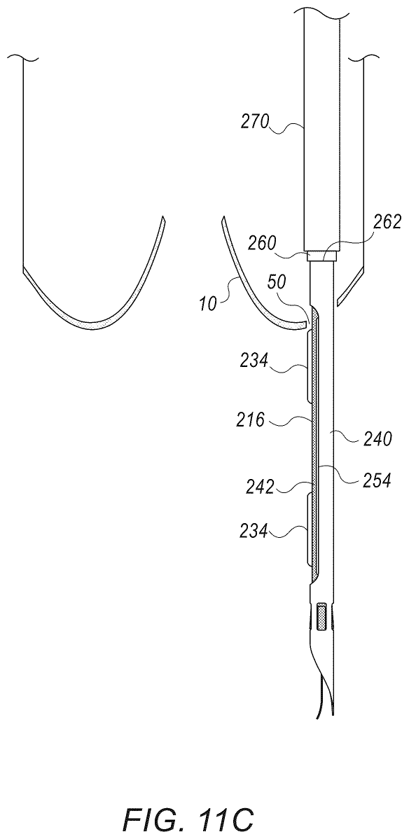

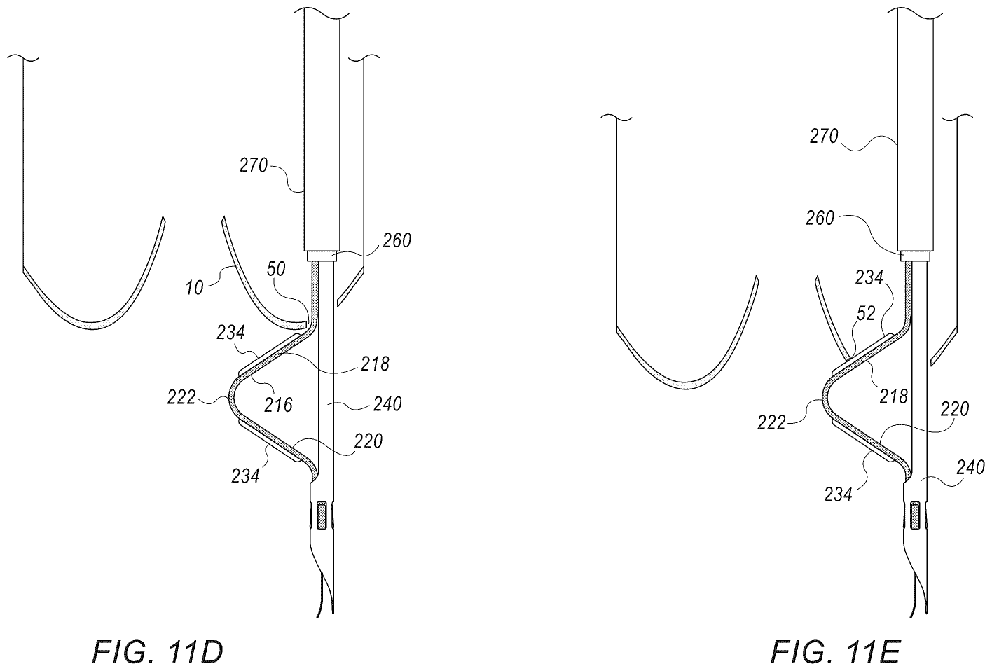

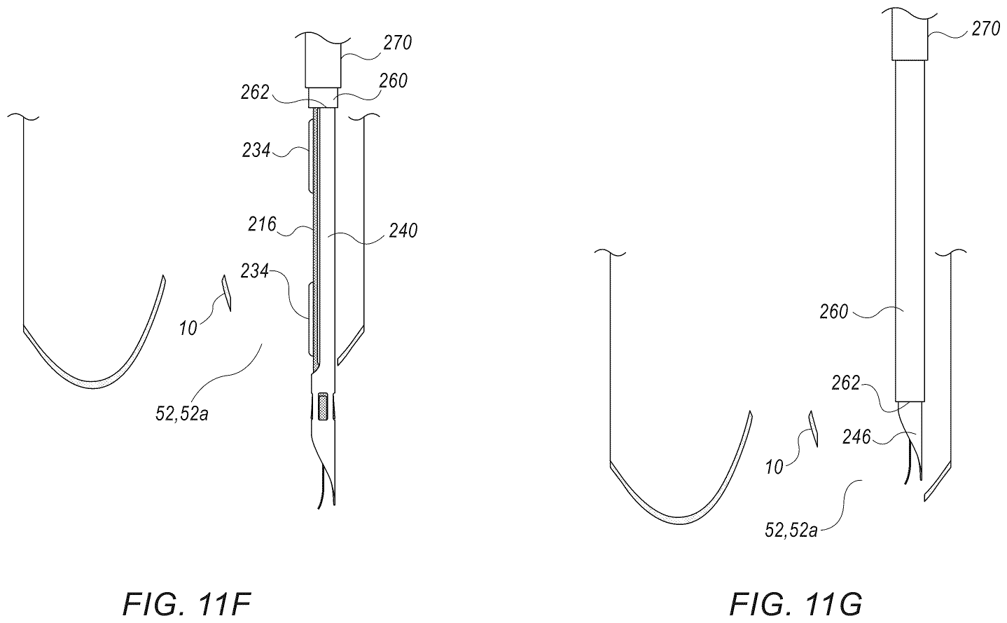

FIGS. 11A-11G illustrate some steps in a method for utilizing a tissue cutting tool 200 for forming an opening within a target tissue. An exemplary implementation of the method is illustrated in FIGS. 11A-11G with respect to forming a leaflet hole 52 inside a host leaflet 10, which can optionally be performed prior to implanting a guest prosthetic valve 100 inside the host valvular structure. The tissue cutting tool 200 can optionally be used to perforate, cut, and/or tear a host leaflet 10, such as a native leaflet 30 or a prosthetic valve leaflet 114 of a previously implanted prosthetic valve.

In some examples, the guidewire 280 can optionally be used as a lacerating member for forming an initial pilot puncture 50. In such examples, the guidewire 280 can optionally be a relatively stiff wire having a distal tip 282 configured to pierce the host leaflet 10 when the guidewire 280 is pressed against the leaflet. In some examples, the guidewire 280 can optionally include a radio-frequency (RF) energy delivery tip 282 to assist with penetration through the leaflet tissue. For this purpose, a suitable RF energy device may optionally be coupled to the guidewire 280, and the RF energy device can apply the RF energy to the guidewire tip 282 to penetrate the host leaflet 10. In any examples disclosed herein wherein a guidewire is used to puncture a leaflet, the guidewire can optionally be coupled to a source of RF energy that applies RF energy to the tip of the guidewire. When the guidewire 280 is used to pierce the leaflet 10, the actuation tube distal edge 242 and/or actuation tube tip 243 can be not as sharp, configured merely to pass through a pilot puncture 50 formed by the lacerating guidewire 280, or it can include a sharp cutting actuation tube distal edge 242 and/or sharp actuation tube tip 243 that can be used in combination with the guidewire 280 that forms an initial puncture in the leaflet 10. For example, the guidewire 280 can optionally be used to form an initial pilot puncture 50 (see FIG. 11A), after which the actuation tube distal portion 246 can optionally be advanced through the host leaflet 10 to form a slightly larger pilot puncture (see FIG. 11B) for subsequent advancement of the bendable section 216 through the host leaflet 10.

In some examples, the guidewire 280 can optionally be used as a lacerating member without an additional tissue cutting distal edge 242 of the actuation tube 240, such that the guidewire 280 can be utilized as the sole component that forms the pilot puncture 50, allowing actuation tube 240 and blade support tube 202 to pass through the pilot puncture 50, optionally with cover shaft 260 disposed therearound, in a manner similar to that illustrated in FIG. 11B.

In some examples, the guidewire 280 can optionally be used as a lacerating member that can be used in addition to a tissue cutting distal edge 242 and/or tip 243 of the actuation tube 240, such that the guidewire 280 can optionally form an initial puncture via a sharp tip 282 or an RF energy delivery tip 282, as illustrated in FIG. 11A, optionally followed by penetration of the sharp distal edge 242 and/or tip 243 of the actuation tube 240 into the leaflet 10 to form the pilot puncture 50, or a pilot puncture 50 which is greater in size than an initial puncture formed by the guidewire tip 282, as shown in FIG. 11B.

In some examples, the guidewire tip 282 is not necessarily sharp enough or otherwise configured to puncture through the host leaflet 10, in which case the guidewire 280 can optionally be utilized for advancement of the tissue cutting tool 200 toward the host valvular structure 12, but terminate in proximity of the host leaflet 10 without piercing through it (for example, remaining above host leaflet 10 instead of passing through the tissue as shown in FIG. 11A), and the sharp distal edge 242 and/or tip 243 of the actuation tube 240 can optionally be then advanced toward and into the host leaflet 10, to form the pilot puncture 50 in a similar manner to that illustrated in FIG. 11B.

The distal end portion of the tissue cutting tool 200 is configured to be inserted into a patient's vasculature, such as within an ascending aorta, and to be advanced towards the host leaflet 10. Positioning tissue cutting tool 200 relative to the host leaflet 10 may optionally comprise advancing tissue cutting tool 200 toward the host leaflet 10 via guidewire 280. During delivery through the patient's vasculature, the bendable section 216 can optionally be concealed within the cover shaft 260 and/or catheter 270, so as to protect the native tissue from being contacted by the relatively sharp edges 236 of the blades 234. The catheter 270 can optionally be utilized to steer the tissue cutting tool 200 towards the desired region of perforation of the target tissue.

Optionally, the actuation tube distal edge 242 and/or actuation tube tip 243 can be similarly concealed within the cover shaft 260 and/or catheter 270, being positioned proximal to the cover shaft distal edge 262 and/or catheter distal edge 274 during delivery, as shown in FIG. 11A, to avoid damage that may be caused to internal anatomical structures of the patient's body due to accidental contact with an optionally sharp actuation tube distal edge 242 and/or tip 243. The actuation tube distal portion 246 can be then optionally pushed toward and through the host leaflet 10 to form the pilot puncture 50 as shown in FIG. 11B.

As shown in FIG. 11B, the actuation tube distal edge 242 can optionally be advanced past cover shaft distal edge 262 prior to advancement through the leaflet 10, either by distally pushing the actuation tube 240 relative to the cover shaft 260, or by retracting the cover shaft 260 relative to the actuation tube 240, but the bendable section 216 can optionally remain concealed within cover shaft lumen 264 during advancement through the leaflet 10. Since the support tube distal portion 208 can optionally be attached to actuation tube distal portion 246, axial advancement of the actuation tube 240 through the host leaflet 10 will cause, in such cases, axial advancement of the blade support tube 202 therewith. Optionally, the cover shaft 260 can be similarly advanced along with the actuation tube 240 through the host leaflet 10, leaving the actuation tube distal edge 242 and/or tip 243 exposed for forming the pilot puncture 50, but retaining the bendable section 216 optionally concealed within cover shaft lumen 264 during advancement through pilot puncture 50. As further shown, advancement of the actuation tube 240 and/or cover shaft 260 through host leaflet 10 can optionally be performed by axially moving the actuation tube distal edge 242 and/or cover shaft distal edge 262 past and distally away from catheter distal edge 274, such that the catheter distal edge 274 remains above the host leaflet 10 while the actuation tube distal edge 242 and/or cover shaft distal edge 262 are positioned below the host leaflet 10.

FIG. 11C shows an optional subsequent step in which the cover shaft 260 is retracted to expose the bendable section 216. As shown, advancement of the actuation tube 240 and blade support tube 202, in an unbent configuration of the bendable section 216, can optionally be performed such that the bendable section 216 is positioned past the host leaflet 10, for example distal to pilot puncture 50.

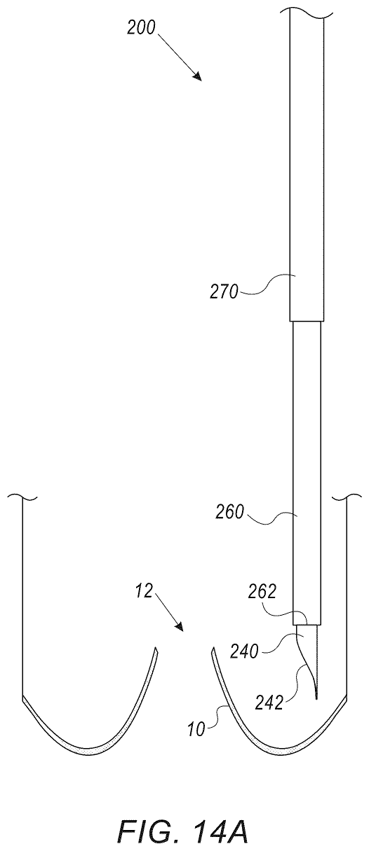

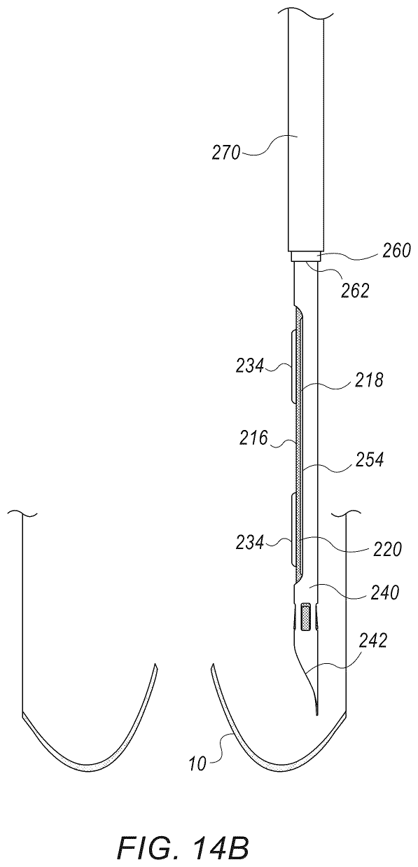

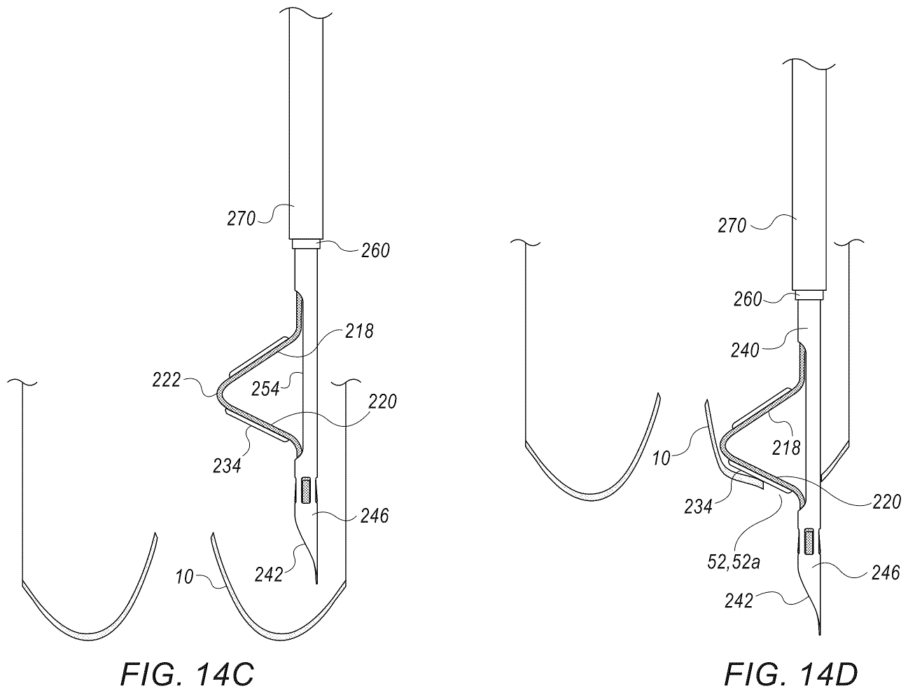

While a portion of the cover shaft 260 disposed over bendable section 216 can be mostly retained within catheter lumen 272 prior to advancing the actuation tube 240 and cover shaft 260 through the host leaflet 10, in some examples, the catheter 270 can optionally be retracted relative to the cover shaft 260 to position the catheter distal edge 274 proximal to the region of the bendable section 216 within the cover shaft 260, prior to advancement of the actuation tube 240 and/or cover shaft 260 through the host leaflet 10, in a manner similar to that illustrated in FIG. 14A, which can be then optionally followed by further advancement of the actuation tube 240 and/or cover shaft 260 through the host leaflet 10 to the position shown in FIG. 11B.

While a portion of the cover shaft 260 is shown to conceal the bendable section 216 up to the moment of positioning the bendable section 216 past the pilot puncture 50 in the example illustrated in FIG. 11B, it is to be understood that in some examples, the cover shaft 260 can optionally be retracted, either after retraction of catheter 270 or simultaneously with catheter 270, prior to advancement of the bendable section 216 through pilot puncture 50, in a manner somewhat similar to that illustrated in FIG. 14B, after which the actuation tube 240 and the bendable section 216, in the unbent configuration, can optionally be advanced through and past the host leaflet 10.

FIG. 11D shows a subsequent step of transitioning the bendable section 216 from the unbent configuration of FIG. 11C to a bent configuration, such as by pushing the blade support tube 202 distally while maintaining the actuation tube 240 in position, by pulling the actuation tube 240 while maintaining the blade support tube 202 in position, or by simultaneously pulling the actuation tube 240 and pushing the blade support tube 202 relative to each other. In the bent configuration, both arms 218, 220 extend farther away from cutout 254, such that at least one of the blades 234 optionally projects on an uncut portion of the target tissue, such as host leaflet 10. In the illustrated example, the blade 234 attached to the proximal arm 218 is closer to the host leaflet, such that upon axially pulling the bendable section 216 relative to and through host leaflet 10, as shown in FIG. 11E, the blade 234 of the proximal arm 218 cuts through the tissue, thereby forming tissue opening or leaflet opening 52.

While the blade 234 closer to host leaflet 10, attached to proximal arm 218, is illustrated to remain completely past and spaced away from the host leaflet 10 during transitioning from the unbent to the bent configuration in FIG. 11D, it is to be understood that in some examples, at least a portion of the blade 234 can optionally move closer and optionally through the host leaflet 10 during transitioning to the bent configuration, thereby starting to cut through the leaflet 10 and optionally starting to form at least a portion of leaflet opening 52 during the transition to the bent configuration. That is to say, the transition between an unbent configuration, such as shown in FIG. 11C, to the bent configuration, can optionally directly cut through at least a portion of the host leaflet 10, similar to the state shown in FIG. 11E, even prior to pulling the bendable section 216 proximally through the host leaflet 10.

Once the leaflet opening 52 has been fully formed inside the host leaflet 10, the bendable section 216 can optionally transition back to the unbent configuration, as shown in FIG. 11F, such as by pulling the blade support tube 202 proximally while maintaining the actuation tube 240 in position, by pushing the actuation tube 240 distally while maintaining the blade support tube 202 in position, or by simultaneously pushing the actuation tube 240 and pulling the blade support tube 202 relative to each other. In some examples, the leaflet opening formed by the tissue cutting tool 200 is a bounded opening 52a that does not form a full tear reaching the leaflet's edge.

While tissue cutting tool 200 is illustrated to include at least two blades 234, one attached to each of the arms 218, 220, it is to be understood that in some examples, only one of the arms can optionally be equipped with a blade 234 attached thereto, while the other arm can be devoid of a blade, and optionally devoid of a retention portion 224 to which a blade can be coupled. In some examples, a blade 234 can be coupled only to the proximal arm 218, while the distal arm 220 can be devoid of a blade (examples not shown explicitly). In such examples, forming a tissue opening or leaflet opening 52 can optionally be performed in a similar manner to that described above with respect to FIGS. 11A-11F. That is to say, the bendable section 216 can optionally transition to the bent configuration while positioned completely or at least partially distal to the host leaflet 10, and then pulled in a proximal direction, such that the blade 234 attached to the proximal arm 218 serves to cut through the host leaflet 10 and form leaflet opening 52.

In some examples, such as when both arms 218, 220 include blades 234 attached thereto, the bendable section 216, while being in the bent configuration, can optionally be passed more than one time through the host leaflet 10, such as by being pulled in a proximal direction to allow the blade 234 attached to the proximal arm 218 to cut through the tissue, as shown in FIGS. 11D-11E, and then translated back in a distal direction through the leaflet, so as to allow the blade 234 attached to the distal arm 220 to further cut through the host leaflet 10. This back-and-forth movement of the bendable section 216 can optionally be repeated for several times. Passing both blades 234 through the tissue can serve to ensure a full cut has been formed, or to expand a cut, so as to form a leaflet opening 52 having a desired size.

While both blades 234 on arms 218, 220 are shown to be similarly sized, in some examples, a blade 234 attached to one arm can optionally be longer or shorter than a blade 234 of the other arm. For example, the blade 234 attached to the distal arm 220 can optionally be longer than the blade 234 attached to the proximal arm 218, such that when the bendable section 216 is translated in a proximal direction through the host leaflet 10 as shown in FIG. 11E, the blade 234 of the proximal arm 218 forms a first cut having a length corresponding to the projected length of the blade on the leaflet 10, and when the bendable section 216 is translated thereafter in the distal direction, in a manner similar to that illustrated in FIG. 14D, the longer blade 234 of the distal arm 220 elongates the cut due to the longer projected length of the blade on the leaflet 10. In some examples, the leaflet opening formed by the tissue cutting tool 200 is a bounded opening 52a that does not form a full tear reaching the leaflet's edge.

While a single blade 234 is shown to be attached to each of the arms 218, 220 throughout the illustrations, it is to be understood that in some examples, any of the arms 218 and/or 220 can optionally be coupled to more than one blade 234.

Passing any blade 234 through the host leaflet 10 will form an elongated cut therethrough. In some examples, the bendable section 216 can optionally be passed through the host leaflet 10 to form a first cut, then rotated around the central longitudinal axis of the tissue cutting tool 200 (the central longitudinal axis is the axis passing along the length of guidewire 280, for example), and passed again through the host leaflet 10, to form another longitudinal cut that can cut through the first cut and be angled relative thereto. For example, forming a first cut by a blade 234 can optionally be followed by a 90-degree rotation of the bendable section 216, such that the second cut, when combined with the first cut, together may form a “+” shaped cut. Other patterns are available, such as by rotating at any other angle and passing the blades of the bendable section 216 through the host leaflet 10 several times, to form a cut in the shape of an asterisk with several longitudinal cuts extending from a central pilot puncture, together forming the leaflet opening 52.

In some examples, the bendable section 216 can optionally be translated in a proximal direction through the host leaflet 10 as shown in FIG. 11E, allowing the blade 234 of the proximal arm 218 to form a first cut, followed by rotating the tissue cutting tool 200 and translating it in a distal direction, allowing the blade 234 of the distal arm 220 to form a second cut which crosses, and is angled relative to, the first cut. The tissue cutting tool 200 can be then optionally rotated again and the procedure can be repeated for as many times as desired.

In some examples, such as when only the proximal arm 218 includes a blade 234 attached thereto, the bendable section 216 can optionally be translated in a proximal direction through the host leaflet 10 as shown in FIG. 11E, allowing the blade 234 of the proximal arm 218 to form a first cut, followed by translating the bendable section 216 in a distal direction back through and past the host leaflet 10, rotating the bendable section, and pulling it back in the proximal direction through the host leaflet 10 to form a second cut which crosses, and is angled relative to, the first cut. The tissue cutting tool 200 can be optionally pushed back through and past the host leaflet 10 and rotated again, optionally repeating this procedure for as many times as desired.

When the leaflet opening 52 is fully formed, and the bendable section 216 reverts back to the unbent configuration as shown in FIG. 11F, the tissue cutting tool 200 can optionally be pulled away from the host leaflet and retracted from the patient's body. In some examples, subsequent to forming the leaflet opening 52 and transitioning the bendable section 216 to the unbent configuration, the cover shaft 260 can optionally be utilized to re-sheath the bendable section 216, concealing the blades 234 inside the cover shaft lumen 264 to protect the surrounding tissue during retrieval, as shown in FIG. 11G. This can optionally be accomplished by distally pushing the cover shaft 260 relative to the blade support tube 202 and/or proximally pulling the blade support tube 202 relative to the cover shaft 260, until at least all blades 234 are concealed within the cover shaft 260. While the cover shaft 260 is illustrated in FIG. 11G to cover the bendable section 216 while the actuation tube distal edge 242 is shown to remain exposed distal to the cover shaft distal edge, it is to be understood that the cover shaft 260 can optionally extend further in the distal direction relative to the actuation tube 240 so as to cover and conceal the actuation tube distal edge 242 and/or tip 243 as well.

While not shown explicitly, it is to be understood that the cover shaft 260, actuation tube 240 and/or blade support tube 202 can be optionally retained inside the catheter lumen 272 prior to and/or during retraction of tissue cutting tool 200, and/or subsequent to formation of the leaflet opening 52 and transitioning of the bendable section 216 to the unbent configuration, similar to all being covered by catheter 270 shown in FIG. 11A for example. It is to be understood that in some examples, a tissue cutting tool 200 can be optionally provided without a catheter but with a cover shaft 260, in which case the cover shaft 260 can be optionally utilized to deliver and/or steer the tissue cutting tool 200. Similarly, a tissue cutting tool 200 can be optionally provided, in some examples, with a catheter 270 but without a cover shaft, in which case the catheter 270 can be optionally further utilized to conceal the blades 234 and/or actuation tube distal edge 242 during passage of the tissue cutting tool 200 through the patient's vasculature, and/or during selected stages of forming the leaflet opening 52 as described herein with respect to cover shaft 260.

The tissue cutting tool 200 may be configured to form the leaflet opening 52 in any of a variety of host valvular structures 12. In the example of FIGS. 11A-11G, as well as FIGS. 14A-14D described below, the host valvular structure 12 can be the valvular structure 113 of a previously implanted prosthetic valve, such as the prosthetic valve 100a of FIG. 3. In such examples, using the tissue cutting tool 200 as described herein to form the leaflet opening 52 in a previously implanted prosthetic valve may be followed by steps for implanting a guest prosthetic valve 100b within the previously implanted prosthetic valve 100a (for example, via a ViV procedure).

Similarly, the host valvular structure 12 in the examples of FIGS. 11A-11G and/or FIGS. 14A-14D can be a valvular structure of a native heart valve, such as the native aortic valve 20 shown in FIGS. 2A-2B. In such examples, the tissue perforating tool 200 can be configured to cut through a native leaflet 30 of the native aortic valve 20. In other examples, the host valvular structure and/or the native valve may refer to another valve of a patient's heart, such as a mitral valve, a pulmonary valve, or a tricuspid valve.

While illustrated and described above with respect to forming a leaflet opening 52 within a host leaflet 10, it is to be understood that the tissue cutting tool 200 may be configured to form a tissue opening through other tissues in a patient's body. For example, prosthetic devices can be delivered to the left atrium or the left ventricle in a transseptal approach, wherein a delivery apparatus is passed through the vena cava, into the right atrium, and through the interatrial septum tissue. Such delivery approaches require puncturing the interatrial septum. Thus, in some implementations, a tissue cutting tool 200 may optionally be utilized to form an opening through the interatrial septum, for example at the site of the fossa ovalis, which is a region of the septum containing tissue of lesser thickness than is typical of the rest of the septum. Thus, any example of tissue cutting tool 200 described above can optionally be utilized in a manner similar to that described above with respect to FIGS. 11A-11G, or described below with respect to FIGS. 14A-14D, to form a tissue opening, equivalent to leaflet opening 52 described with respect to FIG. 11A-11G or 14A-14D, in a target tissue, equivalent to a host leaflet 10 described with respect to FIG. 11A-11G or 14A-14D.

In some examples, tissue cutting tool 200 can optionally be part of a delivery assembly that includes a delivery apparatus carrying a prosthetic valve (examples not shown explicitly). Similarly, a delivery apparatus that includes a tissue cutting tool according to any example of the current disclosure can optionally be used for implantation of other prosthetic devices aside from prosthetic valves, such as stents or grafts.

A delivery apparatus can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

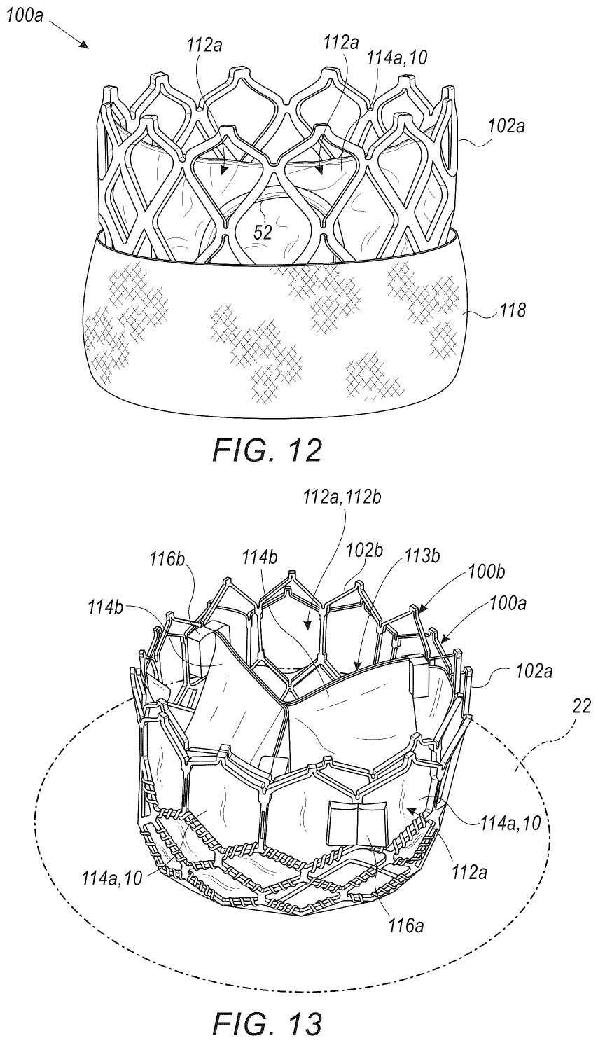

As mentioned, the tissue cutting tools and methods of the current specification can optionally be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 12 shows a previously implanted prosthetic valve 100a subsequent to forming the leaflet opening 52, for example subsequent to the method described above with respect to FIGS. 11A-11G or described below with respect to FIGS. 14A-14D. FIG. 13 shows a configuration in which a second prosthetic valve 100b has been expanded within the leaflet opening 52 of a host prosthetic valve 100a. In the example of FIG. 13, the guest prosthetic valve 100b is the same type of valve as the host prosthetic valve 100a. It is to be understood, however, that ViV procedures may be similarly applied to any other suitable valvular structures, such as different prosthetic valves and/or native heart valves. For example, the guest prosthetic valve 100b need not be the same type of valve as the host prosthetic valve 100a.

In the example of FIG. 12, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 13, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

FIGS. 14A-14D illustrate some steps in a method for utilizing a tissue cutting tool 200 for forming an opening within a target tissue, which can be optionally used in lieu of or in combination with steps of the method described above with respect to FIGS. 11A-11G. The tissue cutting tool 200 can optionally be advanced toward the target tissue in a manner similar to that described above with respect to FIG. 11A. The target tissue is illustrated in FIGS. 14A-14D as a host leaflet 10 of a host valvular structure 12, but it is to be understood that the method described with respect to FIGS. 14A-14D can be optionally implemented to form a tissue opening in any other target tissue, as described above.

As describe above with respect to FIG. 11A, the distal portion of tissue cutting tool 200 can optionally be positioned proximate the host leaflet 10 by advancing the tissue cutting tool 200 through the patient's vasculature over a guidewire (guidewire 280 removed from view in FIGS. 14A-14D for clarity). During delivery through the patient's vasculature, the bendable section 216 can optionally be concealed within the cover shaft 260 and/or catheter 270. The catheter 270 can optionally be utilized to steer the tissue cutting tool 200 towards the desired region of perforation of the target tissue. Optionally, the actuation tube distal edge 242 and/or tip 243 can be similarly concealed within the cover shaft 260 and/or catheter 270, being positioned proximal to the cover shaft distal edge 262 and/or catheter distal edge 274 during delivery, also in a similar manner to that shown in FIG. 11A.

As shown in FIG. 14B, the actuation tube distal edge 242 can be optionally advanced past cover shaft distal edge 262, while still positioned proximal to the host leaflet 10, either by distally pushing the actuation tube 240 relative to the cover shaft 260, and/or by retracting the cover shaft 260 relative to the actuation tube 240. A distal portion of the cover shaft 260, disposed around the bendable section 216, is also shown to be uncovered from the catheter 270 in FIG. 14B. This can be achieved either by axially moving the actuation tube distal edge 242 and/or cover shaft distal edge 262 past and distally away from catheter distal edge 274, and/or retracting the catheter 270 in a proximal direction relative to the cover shaft 260, such that the catheter distal edge 274 is proximal to the cover shaft distal edge 262.

FIG. 14C shows an optional subsequent step in which the cover shaft 260 is retracted to expose the bendable section 216. This can be achieved by distally translating the actuation tube 240 and blade support tube 202, in an unbent configuration of the bendable section 216, relative to the cover shaft 260, and/or retraction of the cover shaft 260 relative to the actuation tube 240 and blade support tube 202, such that the bendable section 216 is positioned distal to the cover shaft distal edge 262 and/or catheter distal edge 274, while still remaining proximal to the host leaflet 10.

While exposure of the bendable section 216 is illustrated in FIGS. 14A-14B to be achieved by first positioning the catheter distal edge 274 proximal to the cover shaft distal edge 262 (FIG. 14A), and then positioning the cover shaft distal edge 262 proximal to the bendable section 216 (FIG. 14B), it is to be understood that the order can be optionally reversed, and that the cover shaft distal edge 262 can be axially translated to a position proximal to catheter distal edge 274 while the bendable section 216 remains concealed within the catheter lumen 272, followed by retraction of catheter 270 and/or advancement of actuation tube 240 and blade support tube 202 to expose the bendable section 216 as shown in FIG. 14B. In some examples, when tissue cutting tool 200 is devoid of a cover shaft, exposure of the bendable section 216 can optionally include exposure thereof out of catheter lumen 272. In some examples, when tissue cutting tool 200 is devoid of a catheter around the cover shaft, exposure of the bendable section 216 can optionally include exposure thereof out of cover shaft lumen 264.

While the actuation tube distal edge 242 is illustrated in FIG. 14A to be exposed while the bendable section 216 remains concealed within cover shaft lumen 262, it is to be understood that both actuation tube distal edge 242 and bendable section 216 can optionally be exposed by continuously extending them out of cover shaft 260 and/or catheter 270 in any manner disclosed above for exposing the bendable section 216.

FIG. 14C shows a subsequent step of transitioning the bendable section 216 from the unbent configuration of FIG. 14B to a bent configuration, such as by pushing the blade support tube 202 distally while maintaining the actuation tube 240 in position, by pulling the actuation tube 240 while maintaining the blade support tube 202 in position, or by simultaneously pulling the actuation tube 240 and pushing the blade support tube 202 relative to each other. In the bent configuration, both arms 218, 220 extend farther away from cutout 254, such that at least one of the blades 234 can optionally project on an uncut portion of the target tissue, such as host leaflet 10. The bendable section 216 is transitioned to the bent configuration in FIG. 14C while still positioned proximal to the host leaflet 10, such that the blade 234 attached to the distal arm 220 is closer to the host leaflet 10. Axially translating the bendable section 216 in a distal direction, toward and through host leaflet 10, as shown in FIG. 14D, allows the blade 234 of the distal arm 220 to cut through the tissue, thereby forming tissue opening or leaflet opening 52.

While the blade 234 closer to host leaflet 10, attached to distal arm 220, is illustrated to remain completely proximal to and spaced away from the host leaflet 10 during transitioning from the unbent to the bent configuration in FIG. 14C, it is to be understood that in some examples, at least a portion of the blade 234 can optionally move closer and optionally through the host leaflet 10 during transitioning to the bent configuration, thereby starting to cut through the leaflet 10 and optionally starting to form at least a portion of leaflet opening 52 during the transition to the bent configuration. That is to say, the transition between an unbent configuration, such as shown in FIG. 14B, to the bent configuration, can directly cut through at least a portion of the host leaflet 10, similar to the state shown in FIG. 14D, even prior to pushing the bendable section 216 distally through the host leaflet 10.

Once the leaflet opening 52 has been fully formed inside the host leaflet 10, the bendable section 216 can optionally transition back to the unbent configuration, similar to the configuration shown in FIG. 11F, such as by pulling the blade support tube 202 proximally while maintaining the actuation tube 240 in position, by pushing the actuation tube 240 distally while maintaining the blade support tube 202 in position, or by simultaneously pushing the actuation tube 240 and pulling the blade support tube 202 relative to each other.

As mentioned above, only one of the arms can optionally include, in some examples, a blade 234 attached thereto. In some examples, a blade 234 can optionally be coupled only to the distal arm 220, while the proximal arm 218 can be devoid of a blade (examples not shown explicitly). In such examples, forming a tissue opening or leaflet opening 52 can be performed in a similar manner to that described above with respect to FIGS. 14A-14D. That is to say, the bendable section 216 can optionally transition to the bent configuration while positioned completely or at least partially proximal to the host leaflet 10, and then pushed in a distal direction, such that the blade 234 attached to the distal arm 220 serves to cut through the host leaflet 10 and form leaflet opening 52.

In some examples, such as when both arms 218, 220 include blades 234 attached thereto, the bendable section 216, while being in the bent configuration, can optionally be passed more than one time through the host leaflet 10, such as by being pushed in a distal direction to allow the blade 234 attached to the distal arm 220 to cut through the tissue, as shown in FIGS. 14C-14D, and then translated back in a proximal direction through the leaflet, so as to allow the blade 234 attached to the proximal arm 218 to further cut through the host leaflet 10. This back-and-forth movement of the bendable section 216 can optionally be repeated for several times. Passing both blades 234 through the tissue can serve to ensure a full cut has been formed, or to expand a cut, so as to form a leaflet opening 52 having a desired size.

As mentioned above, in some examples, a blade 234 attached to one arm can optionally be longer or shorter than a blade 234 of the other arm. For example, the blade 234 attached to the proximal arm 218 can optionally be longer than the blade 234 attached to the distal arm 220, such that when the bendable section 216 is translated in a distal direction through the host leaflet 10 as shown in FIG. 14D, the blade 234 of the distal arm 220 forms a first cut having a length corresponding to the projected length of the blade on the leaflet 10, and when the bendable section 216 is translated thereafter in the proximal direction, in a manner similar to that illustrated in FIG. 11E, the longer blade 234 of the proximal arm 218 elongates the cut due to the longer projected length of the blade on the leaflet 10.

As further mentioned above, the bendable section 216 can optionally be passed through the host leaflet 10 to form multiple intersecting cuts. In some examples, the bendable section 216 can optionally be translated in a distal direction through the host leaflet 10 as shown in FIG. 14D, allowing the blade 234 of the distal arm 220 to form a first cut, followed by rotating the tissue cutting tool 200 and translating it in a proximal direction, allowing the blade 234 of the proximal arm 218 to form a second cut which crosses, and is angled relative to, the first cut. Optionally, the tissue cutting tool 200 can be then rotated again and the procedure can be repeated for as many times as desired.

In some examples, such as when only the distal arm 220 includes a blade 234 attached thereto, the bendable section 216 can optionally be translated in a distal direction through the host leaflet 10 as shown in FIG. 14D, allowing the blade 234 of the distal arm 220 to form a first cut, followed by translating the bendable section 216 in a proximal direction back through and past the host leaflet 10, rotating the bendable section, and pushing it once again in the distal direction through the host leaflet 10 to form a second cut which crosses, and is angled relative to, the first cut. The tissue cutting tool 200 can be pushed forth through and past the host leaflet 10 and rotated again, repeating this procedure for as many times as desired.

When the leaflet opening 52 is fully formed, and the bendable section 216 reverts back to the unbent configuration similar to that illustrated in FIG. 11F, the tissue cutting tool 200 can optionally be pulled away from the host leaflet and retracted from the patient's body, and following any of the steps described at these stages with respect to FIGS. 11F-11G above.

While a guidewire 280 is not illustrated in FIG. 14A-14D, it is to be understood that a guidewire 280 can optionally be utilized in a similar manner to that described above with respect to FIGS. 11A-11G. For example, a guidewire 280 can optionally be utilized to form a pilot puncture 50 through the host leaflet 10, in a manner similar to any of the examples described above with respect to FIG. 11D. An actuation tube cutting distal edge 242 and/or tip 243 can optionally be utilized to form a pilot puncture 50 which can be larger in size than the puncture formed by a guidewire 280, or can optionally be used to form a pilot puncture 50 instead of the guidewire, for example when the guidewire is not configured to puncture through the host leaflet 10. Similarly, the actuation tube distal edge 242 and/or tip 243 can optionally be a blunt or otherwise non-cutting edge and/or a non-penetrating tip, relying for example on a guidewire 280 for forming the pilot puncture 50.

In some examples, when the target tissue is a host leaflet 10 of a host valvular structure 12, a guest prosthetic valve 100 can optionally be positioned in the valvular structure 12 in a compressed state thereof, and expanded therein to implant the guest prosthetic valve 100 inside the host valvular structure. In some examples, the guest prosthetic valve 100 can optionally be positioned inside a leaflet opening 52 in the compressed state of the valve, and expanded therein in a manner that modifies the host leaflet 10. Radially expanding the guest prosthetic valve 100 can be performed in any suitable manner, such as using any suitable valve expansion technique and/or mechanism that is known to the art. In some examples, radial expansion of the guest prosthetic valve 100 can be optionally achieved by inflating an inflatable balloon on which the guest prosthetic valve is mounted. In some examples, radial expansion of the guest prosthetic valve 100 can be optionally achieved by actuating a mechanical actuator of the guest prosthetic valve to mechanically expand a frame of the guest prosthetic valve.

In some examples, the guest prosthetic valve can optionally be a self-expandable prosthetic valve that can be retained during delivery toward the host valvular structure in a capsule or other restraint disposed therearound, and valve expansion can be optionally achieved by removing the capsule or other restraint from the guest prosthetic valve to allow it to radially self-expand within the host valvular structure.

With the guest prosthetic valve received within the leaflet opening 52, radial expansion thereof can serve to increase a size of the leaflet opening and/or to tear the leaflet. As a result, the valve's radial expansion can serve to modify the host leaflet 10 such that the leaflet does not obstruct a cell opening in a frame of the guest prosthetic valve or at least increases the exposed area of the host valvular structure and the guest prosthetic valve that is not covered or obstructed by the modified host leaflet 10 to permit access and sufficient perfusion to the adjacent coronary artery.

While the methods disclosed herein can refer to forming a leaflet opening 52 in a host leaflet 10, prior to positioning and expanding a prosthetic valve 100, it is to be understood that any of the methods can optionally comprise, in some examples, repeating one or more steps disclosed throughout the current specification to form a plurality of cuts and openings in the host valvular structure. For example, steps described above with respect to FIGS. 11A-11G and/or 14A-14D can be performed for forming a first leaflet opening in a first host leaflet, after which the tissue cutting tool 200 can optionally be retracted from the first host leaflet and steered toward another host leaflet, after which the same or equivalent steps can optionally be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be beneficial in examples in which the frame of a host prosthetic valve extends axially in a downstream direction beyond one or both of the coronary arteries when the guest prosthetic valve is implanted within a native heart valve.

Specifically, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In other examples, however, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. In some examples, the host valvular structure can optionally be modified such that the guest prosthetic valve can optionally be implanted by being expanded in a leaflet opening of a first host leaflet that faces the left coronary artery, and such that the second leaflet opening can optionally be formed in a second host leaflet that faces the right coronary artery (or vice-versa).

In some examples, forming the first leaflet opening can optionally be performed prior to forming the second leaflet opening. In other examples, forming the second leaflet opening can optionally be performed prior to forming the first leaflet opening. In some examples, the order of forming leaflet openings is chosen such that the final leaflet opening is formed in the host leaflet in which a guest prosthetic valve 100 is to be positioned and expanded.

It is to be understood that the guest prosthetic valve 100 is not limited to being implanted within an opening 52 of a leaflet. For example, in cases where the tissue cutting tool 200 forms a full tear in a host leaflet that extends to the coaptation edge of the leaflet, the guest prosthetic valve 100 can optionally be positioned at a location between the leaflets of the host valvular structure 12 and then expanded. In such cases, the opening 52 may provide sufficient open space through which blood may flow into the coronary ostia, and/or through which additional access devices, such as coronary catheters, can pass during future interventional procedures.

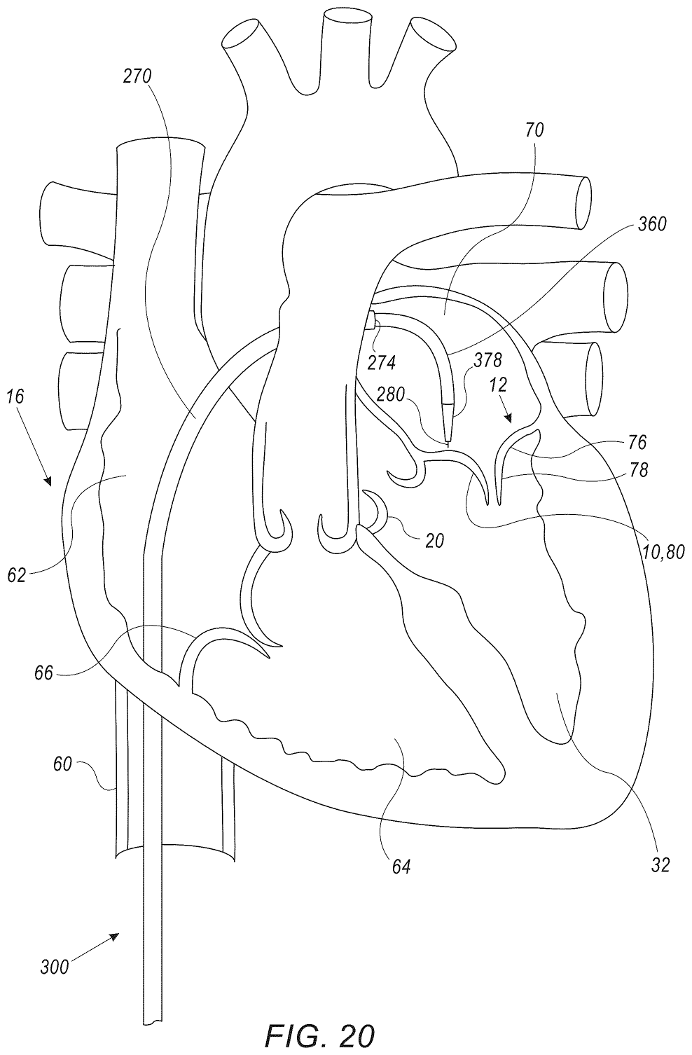

As mentioned above, tissue cutting tools disclosed herein are not limited for use in a native aortic valve or prosthetic valves implanted inside the native aortic valve, but can be rather used for cutting leaflets in other native heart valves, or prosthetic devices implanted in other native heart valve. FIG. 15A illustrates an anatomy of the right-side of a human heart 16. A cross-sectional view showing both the right-side and the left-side of the heart 16 is further illustrated in FIG. 20, for example. The anatomical structure of the heart 16 and blood vessels extending therefrom will be described herein with reference to FIGS. 15A, 15B, and 20 collectively. The heart has a four-chambered conical structure that includes the right atrium 62, the right ventricle 64, the left atrium 70 and the left ventricle 32. The wall separating between the left and right sides of the heart is referred to as the septum 68. The native mitral valve 72 is positioned between the left atrium 70 and the left ventricle 32. The native tricuspid valve 66 is positioned between the right atrium 62 and the right ventricle 64. As described above with respect to FIG. 1, the native aortic valve 20 separates the left ventricle 32 from the ascending aorta 26.

During the diastolic phase, or diastole, deoxygenated blood flows from the right atrium 62 into the right ventricle 64 through the tricuspid valve 66. During systole, leaflets of a normally functioning tricuspid valve 66 close to prevent the venous blood from regurgitating back into the right atrium 62. When the tricuspid valve 66 does not operate normally, blood can backflow or regurgitate into the right atrium 62.

The native mitral valve 72 comprises a mitral annulus 74 and a pair of mitral leaflets 76 extending downward from the annulus 74 and terminating at free edges 11. The leaflets 76 of the mitral valve 72 include an anterior leaflet 80 and a posterior leaflet 78. When operating properly, the anterior leaflet 80 and the posterior leaflet 78 function together as a one-way valve to allow blood flow from the left atrium 70 to the left ventricle 32. Specifically, during diastole, when the muscles of the left atrium 70 and the left ventricle 32 dilate, oxygenated blood flows from the left atrium 70, through the mitral valve 72, into the left ventricle 32. During systole, when the muscles of the left atrium 70 relax and the left ventricle 32 contracts, the blood pressure within the left ventricle 32 increases so as to urge to two mitral leaflets 76 to coapt, thereby preventing blood flow from the left ventricle 32 back to the left atrium 70. At the same time, contraction of the left ventricle 32 forces the oxygen rich blood through the aortic valve 20 and aorta 26 into the body through the circulatory system.

Valvular heart disease can affect functioning of native heart valves, including the mitral valve 72. Mitral regurgitation occurs when the native mitral valve 72 fails to close properly and blood flows back into the left atrium 70 from the left ventricle 32 during systole. Possible causes of this include leaflet prolapse, dysfunctional papillary muscles, issues with chordae tendineae, and/or stretching of the mitral valve annulus due to left ventricle dilation.

A prosthetic valve 100 can be implanted in a malfunctioning native heart valve to prevent or inhibit blood backflow. FIG. 15B shows an exemplary prosthetic valve, which can be similar to any example of a prosthetic valve 100 described above, implanted in a native mitral valve 72. In some instances, as demonstrated in FIG. 15B, a prosthetic valve 100 implanted in the native mitral valve 72 may displace the anterior leaflet 80 towards and into the LVOT 46, which may at least partly obstruct the LVOT 46, thereby reducing blood flow leaving the heart towards the aorta 26.

To avoid reduction of blood flow through a narrowed LVOT 46, the anterior leaflet 80 can be modified by components of a tissue cutting tool 300 prior to implantation of a prosthetic valve 100 within the native mitral valve 72. In some examples, the anterior leaflet 80 is modified by cutting or splitting at least a portion thereof using the cutting tool 300. The modification thus disrupts the impermeable obstruction that would otherwise be formed by the anterior leaflet 80, thereby allowing blood to cross uncovered cell openings 112 of the prosthetic valve, towards the LVOT 46 and aorta 26.

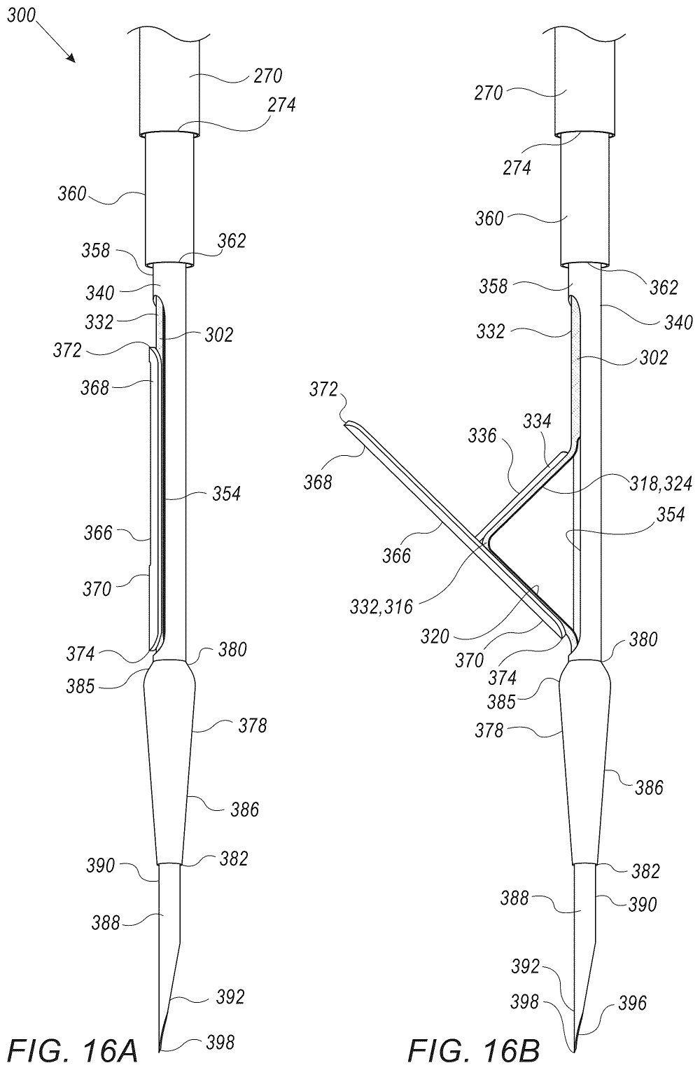

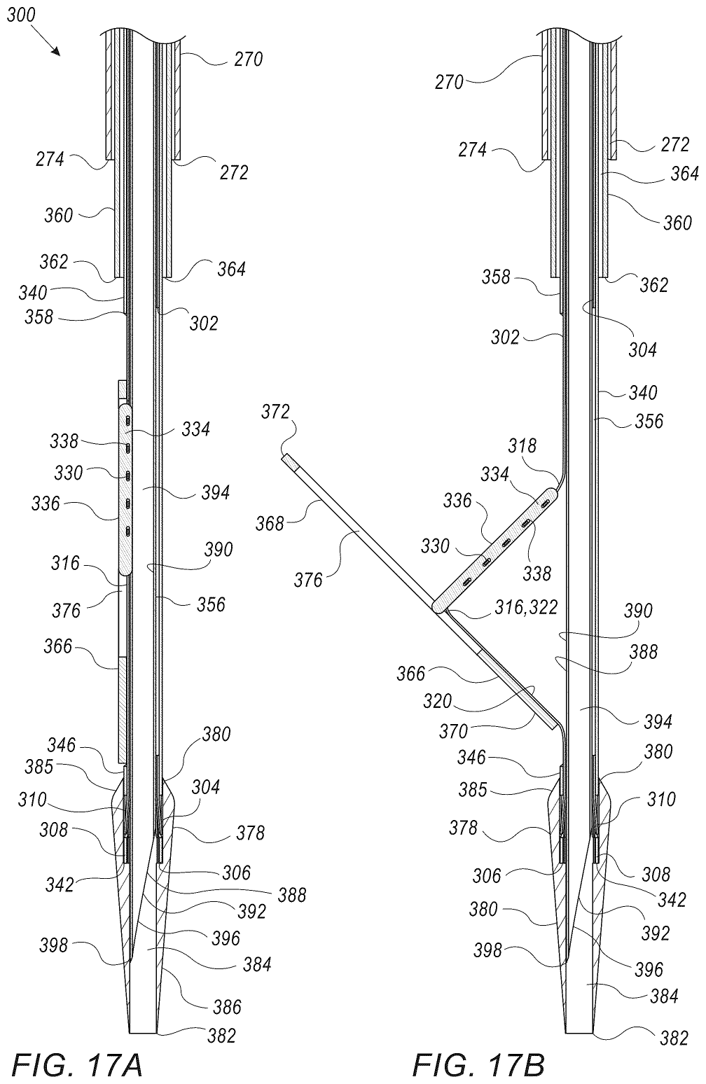

FIGS. 16A-17B show an exemplary tissue cutting tool 300 that can optionally be used to cut a tissue, such as a host leaflet 10 of a host valvular structure 12. The tissue cutting tool 300 comprises a blade support tube 302 having a bendable section 316 to which a blade 334 is attached, wherein the bendable section 316 is configured to transition between bent and unbent configurations. FIGS. 16A and 16B show perspective side views of a distal portion of the tissue cutting tool 300 in unbent and bent configurations, respectively, of its bendable section 316. FIGS. 17A and 17B show cross-sectional side views of the tissue cutting tool 300 in the configurations illustrated in FIGS. 16A and 16B, respectively.

The blade support tube 302 can optionally define a support tube lumen 304, and optionally include a support tube distal portion 308 extending distally to the bendable section 316, and a support tube proximal portion 332 extending proximally from the bendable section 316. The support tube distal portion 308 and support tube proximal portion 332 can optionally have a circular or tubular cross-section, while the bendable section 316 optionally does not enclose a full circular perimeter in cross-section, but can be rather formed with a cutout such that the cross-sectional shape of the bendable section 316 forms an arc to allow it to easily transition to and from a bent configuration. In some examples, the support tube lumen 304 can optionally be sized to allow axial passage of a guidewire needle 388 therethrough.

The tissue cutting tool 300 can optionally further comprise an actuation tube 340 disposed around the blade support tube 302. The actuation tube 340 defines an actuation tube lumen 356, and includes an actuation tube distal portion 346 optionally coupled to the support tube distal portion 308. The actuation tube distal portion 346 comprises an actuation tube distal edge 342 which can optionally be, in some examples, a blunt or atraumatic actuation tube distal edge 342.

The actuation tube 340 can optionally further comprise an actuation tube proximal portion 358 disposed around the support tube proximal portion 332, and a cutout 354 extending between the actuation tube distal portion 346 and the actuation tube proximal portion 358. The cutout 354 can optionally be aligned with the bendable sections 316 of the blade support tube 302, and is configured to expose the bendable section 316 and allow it to protrude radially outward therethrough in the bent configuration, while the actuation tube distal portion 346 extends around the support tube distal portion 308 and the actuation tube proximal portion 358 is disposed around the support tube proximal portion 332. Stated differently, the arms 318, 320, as well as any component attached thereto, such as a blade 334 or a gathering arm 366, are oriented at an angle with respect to the actuation tube 340 in the bent configuration.

The actuation tube lumen 356 can optionally be sized to accommodate the blade support tube 302 therein, and specifically to accommodate the support tube distal portion 308 within the actuation tube distal portion 346 and the support tube proximal portion 332 within the actuation tube proximal portion 358.

The tissue cutting tool 300 can optionally further comprise a cover shaft 360 disposed around the actuation tube 340. The cover shaft 360 defines a cover shaft lumen 364 and terminates at a cover shaft distal edge 362. The actuation tube 340 can optionally be axially movable relative to the cover shaft 360. The cover shaft lumen 364 can optionally be sized to allow passage of the actuation tube 340 and blade support tube 302 therethrough.

In some examples, the tissue cutting tool 300 can optionally further comprise a catheter 270 disposed around the cover shaft 360, which can be similar to the catheter 270 described herein with respect to a tissue cutting tool 300. The cover shaft 360 and actuation tube 340 can optionally be axially movable relative to catheter 270. The catheter lumen 272 can optionally be sized to allow passage of the cover shaft 360 therethrough.

The support tube distal portion 308 terminates at a support tube distal edge 306, which can optionally be either aligned with the actuation tube distal edge 342 as shown, for example, in FIGS. 17A-17B, or can be proximal or distal to the actuation tube distal edge 342. The support tube distal portion 308 can optionally be attached to the actuation tube distal portion 346 in a manner that prevents axial movement therebetween, while the support tube proximal portion 332 and the actuation tube proximal portion 358 can optionally be axially movable relative to each other. Thus, in such cases, axial movement between the support tube proximal portion 332 and the actuation tube proximal portion 358, while the support tube distal portion 308 remain immovable with respect to the actuation tube distal portion 346, serves to transition the bendable section 316 between the unbent and bent configuration.

For example, axially pulling the actuation tube 340 relative to the blade support tube 302, and/or axially pushing the blade support tube 302 relative to the actuation tube 340, serves to transition the bendable section 316 from the unbent configuration of FIGS. 16A and 17A, to the bent configuration shown in FIGS. 16B and 17B. Similarly, axially pulling the blade support tube 302 relative to the actuation tube 340, and/or axially pushing the blade support tube 302 relative to the actuation tube 340, serves to transition the bendable section 316 from the bent configuration of FIGS. 16B and 17B back to the unbent configuration of FIGS. 16A and 17A.

In the bent configuration, the bendable section 316 extends radially away from the longitudinal axis of the tissue cutting tool 300, forming an intermediate bend 322 offset radially farther away from the cutout 354 relative to the unbent configuration. The bendable section 316 includes a proximal arm 318 extending between the support tube proximal portion 332 and the intermediate bend 322, and a distal arm 320 extending between the intermediate bend 322 and the support tube distal portion 308. At least one of the arms 318, 320 can optionally further include a blade 334 attached thereto, the blade 334 having a sharp edge 336 facing radially away from the respective arm 318, 320.

In some examples, only one of the arms is optionally equipped with a blade 334, while the other arm can be devoid of a blade. In some examples, one of the arms 318, 320 is equipped with a blade 334, while a gathering arm 366 is attached to the other one of the arms 318, 320. In some examples, a blade 334 can optionally be attached to the proximal arm 318, and a gathering arm 366 can optionally be attached to the distal arm 320, as illustrated in FIGS. 16A-17B. While a single blade 334 is attached to the proximal arm 318 in the illustrated example, it is to be understood that the proximal arm 318 can optionally include more than one blade 334 attached thereto.

The gathering arm 366 extends between a gathering arm distal end 374 and a gathering arm proximal end 372, and can define a gathering arm distal portion 370 which can optionally be defined, in some examples, as the portion attached to the distal arm 320, and a gathering arm proximal portion 368 which extends proximally from the gathering arm distal portion 370, and can optionally be defined, in some examples, as a portion which is not attached to the distal arm 320. The gathering arm distal portion 370 can optionally extend between the intermediate bend 322 and the gathering arm distal end 374, and the gathering arm proximal portion 368 can optionally extend between the intermediate bend 322 and the gathering arm proximal end 372. In some examples, the gathering arm 366 can optionally be atraumatic or blunted along any of its edges or surfaces.

In the unbent configuration, as shown in FIGS. 16A and 16A, the proximal arm 318 with the blade 334 attached thereto, and the distal arm 320 with the gathering arm 366 attached thereto, can optionally be substantially parallel to the longitudinal axis of the tissue cutting tool 300, while in the bent configuration, as shown in FIGS. 16B and 17B, the proximal arm 318 with the blade 334 attached thereto, and the distal arm 320 with the gathering arm 366 attached thereto, are angled relative to the longitudinal axis of the tissue cutting tool 300 and relative to the remainder of the blade support tube 302 and/or actuation tube 340.

As mentioned above, the gathering arm proximal portion 368 can optionally extend proximally from the distal arm 320 and the intermediate bend 322, and can optionally cover at least a portion of, and in some examples, the entire length of, the blade 334 attached to the proximal arm 318, in the unbent configuration. In the bent configuration, the sharp edge 336 of the blade 334 coupled to the proximal arm 318 can optionally be oriented in a proximal direction, while the gathering arm proximal portion 368 has the same angular orientation as that of the distal arm 320, extending angularly radially away from the proximal arm 318 and the blade 334 attached thereto.

Each of the blade 334 and the gathering arm 366 can optionally be coupled to the proximal arms 318 and distal arm 320, respectively, by any suitable means, such as by gluing, welding, and the like. The blade support tube 302, or at least the bendable section 316 thereof, can optionally be made from a flexible material that can transition between bent and unbent configurations. In some examples, the blade support tube 302, or at least the bendable section 316 thereof, can optionally be made of a shape memory material, such as Nitinol. In some examples, the blade 334 can optionally be coupled to the proximal arm 318 in a similar manner to that described above with respect to FIGS. 6-8 for coupling a blade 234 to any arm 218, 220 of cutting tool 200. For example, the proximal arm 318 can optionally include a retention portion 324 comprising a longitudinal slot with a plurality of protrusion 330 extending into matching side openings 338 of the blade 334.

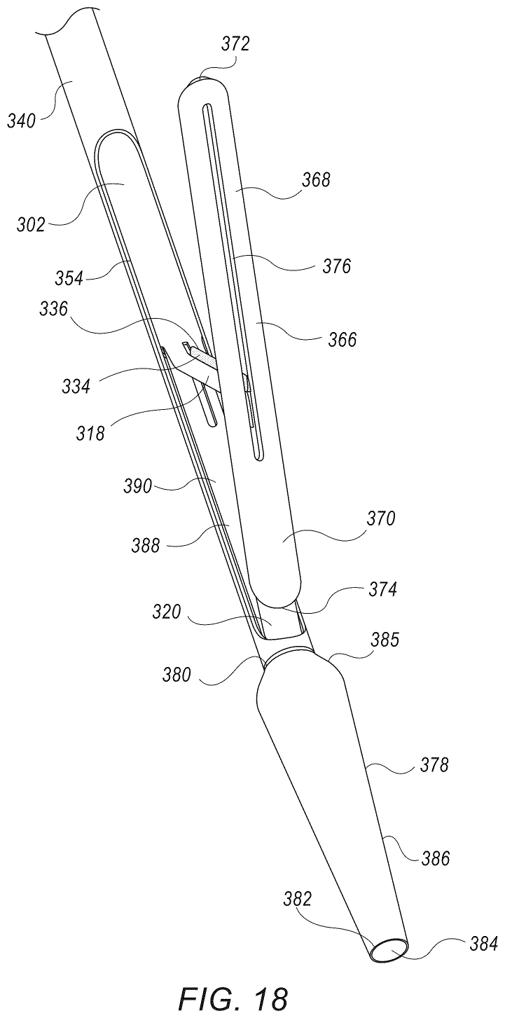

FIG. 18 shows a perspective view of a distal portion of an exemplary tissue cutting tool 300, in the bent configuration corresponding to that shown in FIG. 16B. Optionally, the gathering arm 366 can further include a gathering arm slot 376, as shown in FIG. 18. The gathering arm slot 376 can optionally extend longitudinally along a portion of the gathering arm 366 congruent with the blade 334, such that when the gathering arm proximal portion 368 extends over the proximal arm 318 in the unbent configuration, the blade 334 can extend into the gathering arm slot 376. In some examples, the thickness of the gathering arm 366, at least at the region of the slot 376, can optionally be equal to or greater than the radial extension of the blade 334 away from the proximal arm 318.

In some examples, the tissue cutting tool 300 can optionally further comprise a dilator 378 that can optionally be conical or frustoconical in shape, and extend between a dilator proximal end 380 and a dilator distal end 380. The dilator can optionally include a dilator distal tapering surface 386 terminating at the dilator distal end 382. Optionally, the dilator 378 can further define a dilator proximal tapering surface 385 extending between the dilator distal tapering surface 386 and the dilator proximal end 380. The dilator can optionally be attached to the actuation tube distal portion 346 and define a dilator lumen 384 which is open ended at the dilator distal end 382. The dilator lumen 384 can optionally be continuous with the support tube lumen 304.

Attachment of the actuation tube 340 to the dilator 378 can optionally be achieved by a variety of methods, such as overmolding, radio-frequency welding, through an adhesive, and/or a combination thereof. In some examples (not illustrated), the actuation tube 340 can optionally extend through the entire length of the dilator 378, such that the actuation tube distal edge 342 can optionally be aligned with the dilator distal end 382. In some examples (not illustrated), the actuation tube 340 can optionally be coupled to one or more components, such as collars or other connectors, which are in turn attached to the dilator 378. In some examples (not illustrated), the blade support tube 302 can optionally extend through the entire length of the dilator 378, such that the support tube distal edge 306 is aligned with the dilator distal end 382.

In some examples, the tissue cutting tool 300 can optionally further comprise a hollow needle 388 extending through the support tube lumen 304. The needle 388 can optionally comprise a needle head 392 and a needle shaft 390 extending proximally from the needle head 392, collectively defining a needle lumen 394. The needle head 392 is configured to pierce a target tissue, such as a host leaflet 10 of a host valvular structure 12. The needle head 392 can optionally define an angled surface 396 terminating at a sharp needle tip 398 configured to facilitate piercing the host leaflet 10 when the needle 388 is pressed thereagainst. The needle 388 can optionally be axially movable relative to the any of the blade support tube 302, actuation tube 340, and dilator 378. The needle lumen 394 can optionally be sized to allow passage of a guidewire 280 therethrough.

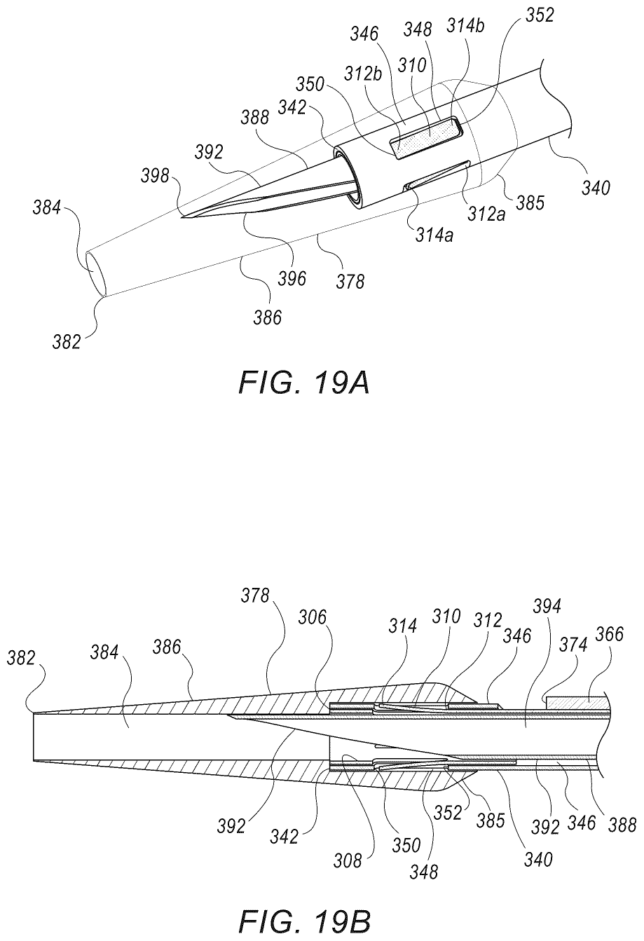

FIGS. 19A-19B illustrate an exemplary manner by which support tube distal portion 308 can optionally be coupled to the actuation tube distal portion 346, which is generally similar to the coupling described above with respect to FIG. 9-10. FIG. 19A is a perspective view of a distal portion of the tissue cutting tool 300, wherein the dilator 378 is shown with transparency for illustrative purpose. FIG. 19B is a cross-sectional side view of the portion of the tool 300 shown in FIG. 19A. An exemplary support tube distal portion 308 can optionally be formed to include a plurality of tabs 310 proximal to the support tube distal edge 306. The tabs 310 can optionally be arranged around the circumference of support tube distal portion 308, wherein each tab 310 extends from a tab base 312 to a tab free end 314. Each tab 310 can be optionally formed by cutting a U-shaped slot, wherein the uncut end forms the tab base 312 which can optionally be integrally formed with the support tube distal portion 308.

The plurality of tabs 310 can optionally include at least two oppositely oriented tabs 310, comprising at least one distally oriented tab 310a with a distal tab free end 314a formed at its distal edge, and at least one proximally oriented tab 310b with a proximal tab free end 314b. Any number of tabs 310 and arrangements thereof is contemplated. The number of distally oriented tabs 310a can be similar to or different than the number of proximally oriented tabs 310b, and the tabs 310 can be equally spaced from each other or unequally spaced from each other in the circumferential direction.

An actuation tube distal portion 346 of a corresponding exemplary actuation tube 340 can optionally include a plurality of windows 348 matching in number the plurality of tabs 310, circumferentially arranged around the circumference of the actuation tube distal portion 346, positioned and sized to accommodate the tabs 310. Each window 348 extends between a window distal edge 350 and a window proximal edge 352. The tabs 310 can optionally be biased radially outward, extending radially away from the tab base 312 to the tab free end 314, such that the tab free end 314 is disposed at the level of the edges 350, 352 of the windows 348.

A distal tab free end 314a of each distally oriented tab 310a is facing a window distal edge 350, while a proximal tab free end 314b of each proximally oriented tab 310b is facing a window proximal edge 352. The distal tab free ends 314a, abutting window distal edges 350, prevent a distally oriented movement of the support tube distal portion 308 relative to the actuation tube distal portion 346, and/or a proximally oriented movement of the actuation tube distal portion 346 relative to the support tube distal portion 308. Similarly, the proximal tab free ends 314b, abutting window proximal edges 352, prevent a proximally oriented movement of the support tube distal portion 308 relative to the actuation tube distal portion 346, and/or a distally oriented movement of the actuation tube distal portion 346 relative to the support tube distal portion 308. Thus, the inclusion of both distally oriented tabs 310a and proximally oriented tabs 310b serves to prevent axial movement between the support tube distal portion 308 and the actuation tube distal portion 346 both in the distal and proximal directions.

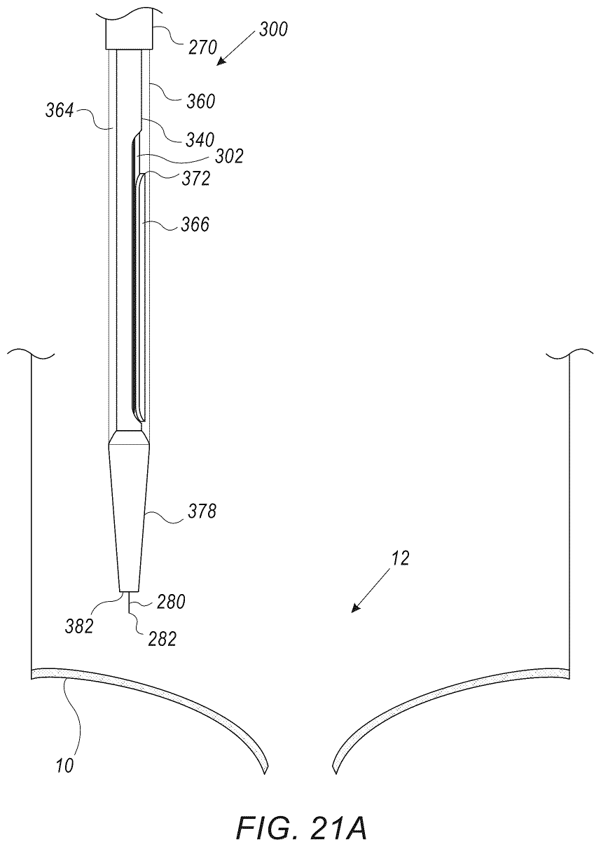

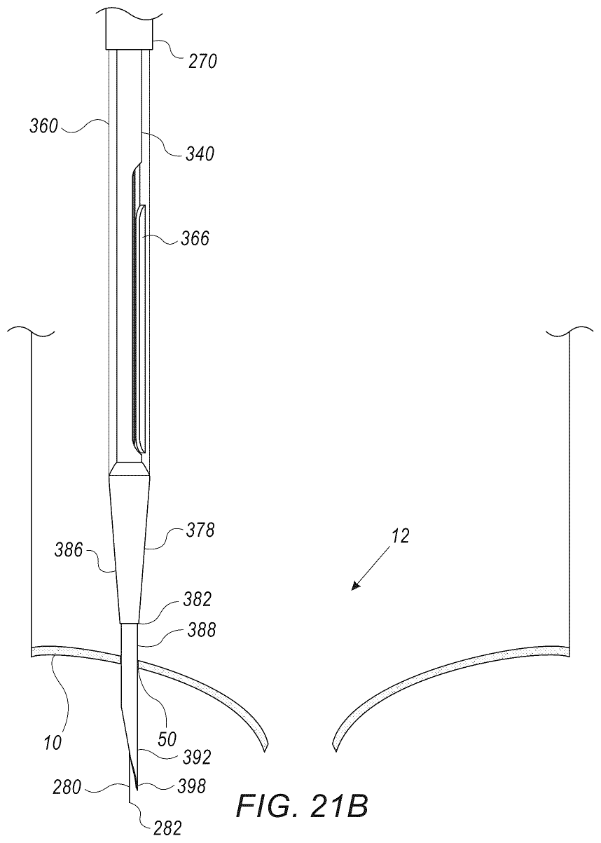

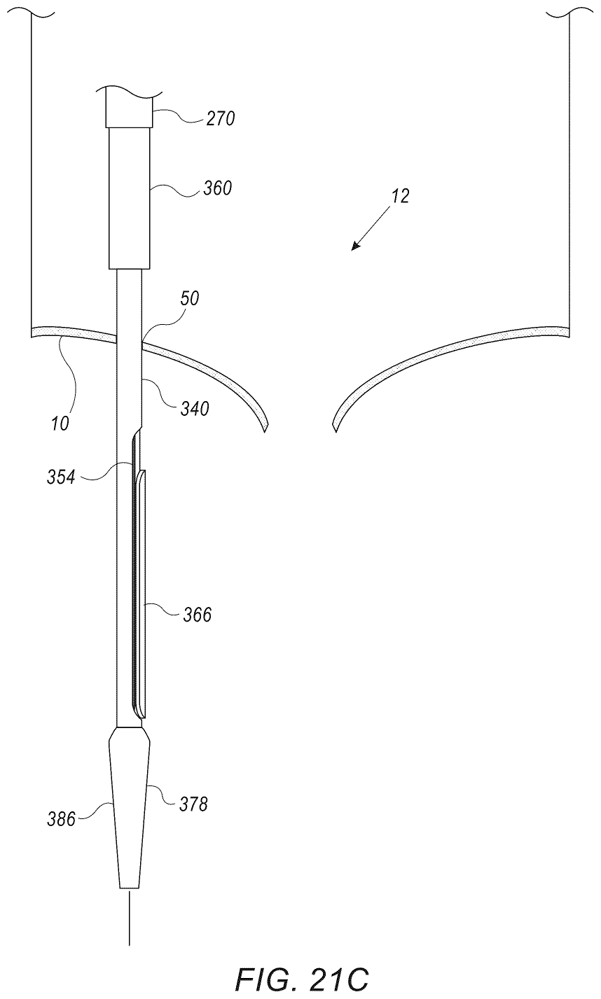

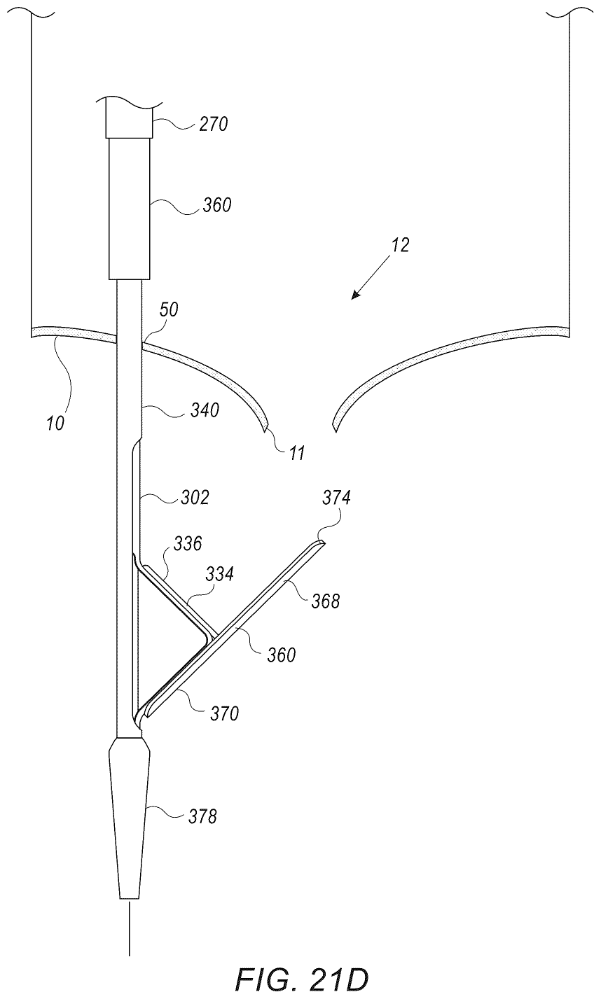

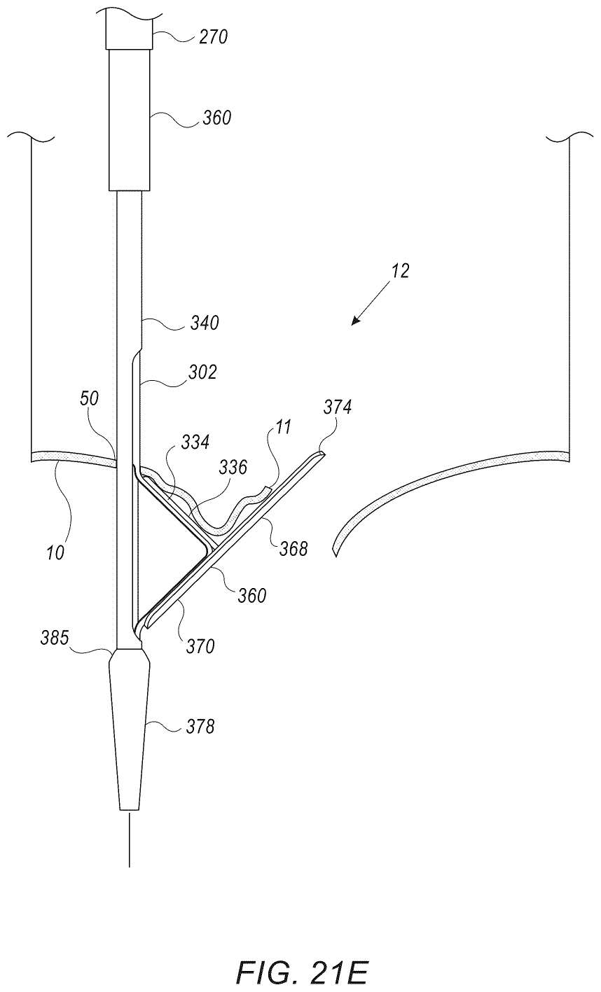

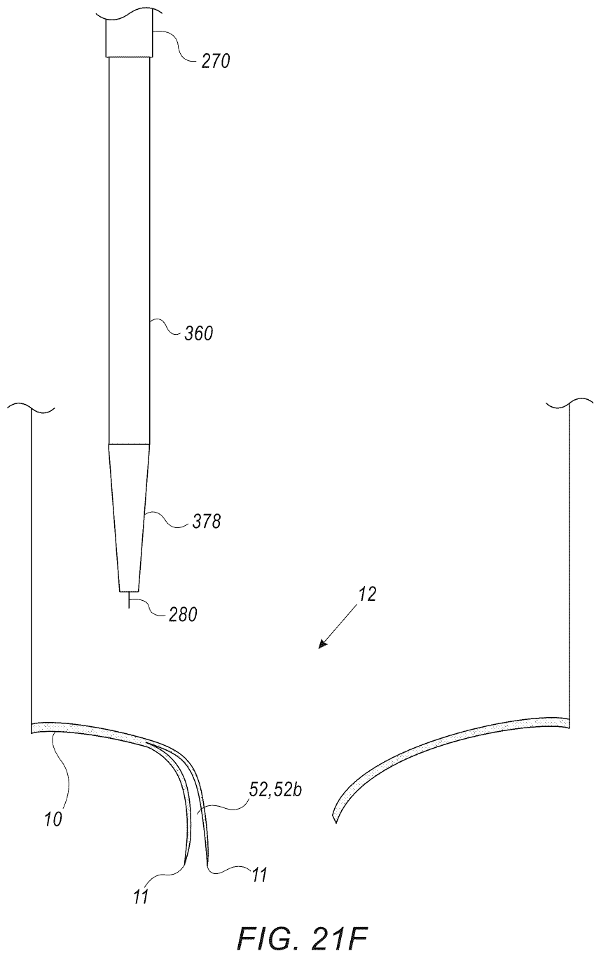

FIGS. 20-21F illustrate some steps in a method for utilizing a tissue cutting tool 300 for forming cutting a target tissue. An exemplary implementation of the method is illustrated in FIGS. 20-21F with respect to cutting the anterior leaflet 38 of a mitral valve 72, which can optionally be performed prior to implanting a prosthetic valve 100 inside the native mitral valve 72.

The distal end portion of the tissue cutting tool 300 is configured to be inserted into a patient's vasculature, such as optionally within the inferior vena cava 60, and to be advanced towards and optionally passed through the interatrial septum 68 of the subject (septum 68 indicated in FIG. 15A). For applications in which cutting tool 300 is used to modify a leaflet 76 of the mitral valve 72 of the patient, the catheter 270 is typically configured for initial advancement through the patient's vasculature into the right atrium 62 and through the septum 68, until the distal end portion of the catheter 270 is positioned in the left atrium 70. The distal end portion of the catheter 270 can be then optionally steered such that it is positioned in a desired spatial orientation within the left atrium 70. Such an optional steering procedure can be performed with the aid of imaging, such as fluoroscopy, transesophageal echo, and/or echocardiography.

In some examples, the tissue cutting tool 300 can optionally be advanced through the vasculature into the right atrium 62 using a suitable point of origin typically determined for a given patient. In some examples, the tissue cutting tool 300 can be optionally introduced into the femoral vein of the patient, through the inferior vena cava 60, into the right atrium 62, and into the left atrium 70 transseptally, typically through the fossa ovalis (hidden from view in FIG. 20). In some examples, the tissue cutting tool 300 can be optionally introduced into the basilic vein, through the subclavian vein to the superior vena cava, into the right atrium 62, and into the left atrium 70 transseptally, typically through the fossa ovalis (not shown). In some examples, the tissue cutting tool 300 can be optionally introduced into the external jugular vein, through the subclavian vein to the superior vena cava, into the right atrium 62, and into the left atrium 70 transseptally, typically through the fossa ovalis (not shown)

In some examples, the needle 388 and dilator 378 of the tissue cutting tool 300 can optionally be further utilized for passing the cutting tool 300 through the septum 68. For example, in order to advance the cutting tool 300 transseptally into the left atrium 70, the dilator 378 can optionally be advanced to the septum 68, and the needle 388 can optionally be pushed from within the dilator 378 and allowed to puncture the septum 68 to create an opening that facilitates passage of the dilator 378 and subsequently remainder of the cutting tool 300 therethrough and into the left atrium 70.

During delivery into the left atrium 70 and towards the mitral leaflet 76, any of the blade support tube 302, actuation tube 340, and/or cover shaft 360, can optionally be at least partially retained inside the catheter 270, while the dilator 378 can optionally be exposed, extending past the catheter distal edge 274, to facilitate smooth passage of the tissue cutting tool 300 through the vasculature and anatomic structures.

The cover shaft 360 is shown in FIGS. 21A-21B with transparency for illustrative purpose. Positioning tissue cutting tool 300 relative to the host leaflet 10, such as the anterior leaflet 80, may optionally comprise advancing tissue cutting tool 300 toward the host leaflet 10 via guidewire 280, as shown in FIGS. 20 and 21A. During delivery through the patient's vasculature, the bendable section 316 can optionally be concealed within the cover shaft 360 and/or catheter 270, so as to protect the native tissue from being contacted by the relatively sharp edge 336 of the blade 334. The catheter 270 can optionally be utilized to steer the tissue cutting tool 300 towards the desired region of cut initiation of the target tissue.

The needle head 392 with the needle tip 398 can optionally be positioned proximal to the dilator distal end 382, so as to conceal it within the dilator lumen 384 or support tube lumen 304, for example, during delivery, to avoid damage that may be caused to internal anatomical structures of the patient's body due to accidental contact with a sharp needle tip 398. The needle 388 can optionally be then pushed toward and through the host leaflet 10 to form the pilot puncture 50 as shown in FIG. 21B, while the remainder of the tissue cutting tool 300 can optionally be still positioned proximal to the host leaflet 10.

When included, the dilator 378 can optionally be inserted into the pilot puncture 50 to expand the pilot puncture 50. As the dilator 378 is inserted into the host leaflet 10, the inherent resiliency of the leaflet 10 may urge the leaflet 10 radially inwardly against the dilator 378. The dilator 378 can optionally have sufficient stiffness to facilitate advancement thereof through the leaflet 10, wherein the gradually tapering shape of the dilator distal tapering surface 386 facilitates expanding the pilot puncture 50 to a greater diameter. The dilator can optionally be further advanced distally to the host leaflet 10, as shown in FIG. 21C. Since the actuation tube 340 can optionally be attached to the dilator 378, and the support tube distal portion 308 can optionally be attached to actuation tube distal portion 346, axial advancement of the dilator 378 through the host leaflet 10, in such cases, will cause axial advancement of the actuation tube 340 and the blade support tube 302 therewith. Advancement may optionally be continued until the entire length of the bendable section 316 and/or cutout 354 is positioned below (e.g., distal to) the host leaflet 10. The needle head 392 can optionally be reconcealed by pulling the needle 388 relative to the dilator 378 and/or pushing the dilation 378 over the needle 388.

As further shown in FIG. 21C, the actuation tube 340 and blade support tube 302 can optionally be uncovered from the cover shaft 360 at the unbent position of the blade support tube 302 distal to the host leaflet 10. In some examples, unsheathing of the actuation tube 340 and blade support tube 302 can optionally be performed by distally advancing the actuation tube 240 distally through the host leaflet 10 while the cover shaft 360 is kept proximal to the host leaflet 10. In some examples, unsheathing of the actuation tube 340 and blade support tube 302 can optionally be performed by exposing the actuation tube 340 and blade support tube 302 out of the cover shaft 360 while they are still proximal to the host leaflet 10, which can be accomplished by distally pushing the actuation tube 340 out of the cover shaft 360 and/or pulling the cover shaft 360 proximally from the actuation tube 340, after which the exposed actuation tube 340 and blade support tube 302 can be advanced through the pilot puncture 50 to the position shown in FIG. 21C. In some examples, unsheathing of the actuation tube 340 and blade support tube 302 can optionally be performed by distally advancing both the actuation tube 340 and the cover shaft 360 through the pilot puncture 50, with the cover shaft 360 still covering the bending section 316 of the support tube 302, after which the cover shaft 360 can be retracted to expose the bending section 316 as shown in FIG. 21C.

FIG. 21D shows a subsequent step of transitioning the bendable section 316 from the unbent configuration of FIG. 21C to a bent configuration, such as by pushing the blade support tube 302 distally while maintaining the actuation tube 340 in position, by pulling the actuation tube 340 while maintaining the blade support tube 302 in position, or by simultaneously pulling the actuation tube 340 and pushing the blade support tube 302 relative to each other. In the bent configuration, both arms 318, 320 extend farther away from cutout 354, such that the blade 334 optionally projects on an uncut portion of the host leaflet 10, and the gathering arm proximal portion 368 optionally extends proximally from the intermediate bend 322 and away from the blade 334, towards the free edge 11 of the host leaflet 10.

Upon axially pulling the tissue cutting tool 300, in the bent configuration of the blade support tube 302, towards the leaflet 10, the blade 334 gets closer to the leaflet 10, while the gathering arm proximal end 372 can optionally extend past the free edge 11 of the leaflet 10, causing the portion of the leaflet 10 extending between the pilot puncture 50 and the free edge 11 to gather inside the space defined between the gathering arm proximal portion 368 and the proximal arm 318 of the blade support tube 302, as shown in FIG. 21E. Continued pulling of the cutting tool 300 will cause the blade 334 to cut through the tissue of the host leaflet 10, thereby forming a leaflet opening 52b in the form of a full cut extending to the free edge 11 of the leaflet 10, as shown in FIG. 21F.

In the absence of a gathering arm 366, the host leaflet 10 could slip relative to the blade 334 as the blade contacts and is proximally pulled against the leaflet 10, due to the orientation and flexibility of the leaflet 10. Advantageously, utilization of a gathering arm 366 to gather the leaflet 10 towards the blade 334 as it is proximally pulled through the leaflet 10, allows the blade 334 to cut all the way to the free edge 11.

Once the leaflet opening 52b, in the form of a full cut, has been fully formed inside the host leaflet 10, the bendable section 316 can optionally transition back to the unbent configuration, such as by pulling the blade support tube 302 proximally while maintaining the actuation tube 340 in position, by pushing the actuation tube 340 distally while maintaining the blade support tube 302 in position, or by simultaneously pushing the actuation tube 340 and pulling the blade support tube 302 relative to each other. The cover shaft 360 can optionally be then utilized to re-sheath the bendable section 316.

While not shown explicitly, it is to be understood that the cover shaft 360, actuation tube 340 and/or blade support tube 302 can optionally be retained inside the catheter lumen 272 prior to and/or during retraction of tissue cutting tool 300, and/or subsequent to formation of the leaflet opening 52b and transitioning of the bendable section 316 to the unbent configuration. Subsequent to completion of the cutting procedure and returning the bendable section 316 to the unbent configuration, the tissue cutting tool 300 can optionally be retrieved from the patient's body.

When the host leaflet 10 is the anterior leaflet 80, this procedure of cutting the anterior leaflet 80 helps prevent the anterior leaflet 80 from obstructing or otherwise interfering with the LVOT 46. After retrieval of the tissue cutting tool 300, a new prosthetic valve 100 can optionally be implanted in the native mitral valve 72, wherein blood can flow through uncovered cell openings 112 of the valve 100 and the cut-out segment or opening 52b of the anterior leaflet 80. In some examples, the tissue cutting tool 300 can optionally be retrieved while the guidewire 280 remains in position, and the new prosthetic valve 100 can optionally be delivered over the guidewire 280.

It is to be understood that in some examples, a tissue cutting tool 300 can optionally be provided without a catheter but with a cover shaft 360, in which case the cover shaft 360 can be optionally utilized to deliver and/or steer the tissue cutting tool 300. Similarly, a tissue cutting tool 300 can optionally be provided with a catheter 270 but without a cover shaft, in which case the catheter 270 can be optionally further utilized to cover distal portions of the blade support tube 302 and/or actuation tube 340 during advancement of the tissue cutting tool 300 through the patient's vasculature, and/or during selected stages of forming the leaflet opening 52b as described herein with respect to cover shaft 360. In some examples, the tissue cutting tool 300 can optionally be provided without any of the cover shaft 360 and catheter 270, or with a catheter that does not necessarily conceal the bendable section 316 of the blade support tube 302 during delivery or retraction stages, relying on the gathering arm 366 optionally sufficiently concealing the blade 334 to protect the surrounding anatomy from being engaged thereby, when the blade support tube is in the unbent configuration.

While a method of utilizing a leaflet cutting tool 300 is described and illustrated herein with respect to cutting an anterior leaflet 80 of the native mitral valve 72, it is to be understood that any method for using leaflet cutting tool 300 can optionally be similarly used for cutting any other tissue or leaflet, such as the posterior leaflet 78 of a native mitral valve 72, a leaflet of a native tricuspid valve 66, an aortic leaflet 31, or a prosthetic leaflet 114 of a previously implanted prosthetic valve. For example, if modification of a leaflet of a tricuspid valve 66 is desired, advancement of a leaflet cutting tool 300 towards the annulus of a tricuspid valve 66 can optionally be performed in a similar manner to that described above with respect to the mitral valve 72, but without penetrating the septum, with the distal end portion of the tool positioned in the right atrium 62, similarly advancing the a needle 388 followed by the actuation tube 340 and unbent blade support tube 302 through a tricuspid leaflet towards the right ventricle 64, moving the bendable section 316 to the bent configuration in the right ventricle 64, and proximally pulling to cut through a portion of the leaflet gathered by the gathering arm 366.

While various exemplary tissue cutting tools 200 disclosed herein can optionally be utilized in a manner that forms a leaflet opening that can be either a bounded opening 52 or a full cut 52b that extends to the free edge 11 of the leaflet 10, a tissue cutting tool 300 that includes a gathering arm 366 will ensure that the leaflet opening is a full cut 52b extending from the point of penetration at the pilot puncture 50 to the free edge 11 of the leaflet 10.

It is to be understood that a dilator 378 is described and shown as part of a tissue cutting tool 300 by way of illustration and not limitation, and that a tissue cutting tool 300 can optionally be provided and utilized without a dilator 378. Furthermore, it is to be understood that any exemplary tissue cutting tool 300 disclosed herein can optionally be provided without a needle 388 extendable through, and relative to, the blade support tube 302. For example, a tissue cutting tool 300 can optionally include other types of lacerating devices, such as a lacerating (e.g., RF conducting) guidewire 280 as described above with respect to optional utilization thereof with a tissue cutting tool 200, or the actuation tube 340 can have a cutting actuation tube distal edge terminating at a sharp tip, similar to a cutting actuation distal edge 242 and actuation tube tip 243 described above. When the actuation tube 340 includes a cutting actuation distal edge and tip, the tissue cutting tool 300 can optionally be devoid of a dilator 378 attached to the actuation tube, to allow exposure of the actuation tube distal edge to pierce the target tissue (e.g., host leaflet 10) thereby.

It is to be understood that any exemplary tissue cutting tool 200 disclosed herein can optionally further include a needle, similar to needle 388, extendable through the support tube lumen 204. In some examples, the actuation tube distal edge 242 is not a cutting edge, and does not necessarily include a sharp or pointed tip. While exemplary tissue cutting tool 200 are described and illustrated herein without a dilator, it is to be understood that any exemplary tissue cutting tool 200 can optionally further include a dilator, similar to dilator 378, which can be attached to the actuation tube 240, in which case the lacerating member of such a tissue cutting tool 200 will not be the actuation tube 240 but rather a lacerating guidewire 280 and/or a needle, similar to needle 388.

While the tissue cutting tool 300 is described and illustrated herein to include a blade 334 attached to the proximal arm 318 and a gathering arm 366 attached to the distal arm 320, it is to be understood that the positions can optionally be reversed, and that the blade 334 can be attached, in some examples, to the distal arm 320, while the gathering arm proximal portion 368 will be attached to the proximal arm 318, in which case the bendable section 316 can be exposed and transition to a bent configuration while positioned above (e.g., proximal to) the host leaflet, and then distally pushed instead of being pulled, in a manner somewhat similar to that illustrated in FIGS. 14A-14D, mutatis mutandis, to form a full cut in a host leaflet 10, which can optionally be, in some examples, an aortic leaflet in which a full cut formation is desired.

Any of the tools, devices, apparatuses, etc. herein can optionally be sterilized (for example, with heat, radiation, and/or chemicals, etc.) to ensure they are safe for use with patients, and any of the methods herein can optionally include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A tissue cutting tool comprising: an actuation tube comprising: an actuation tube distal portion; and an actuation tube proximal portion; and a blade support tube comprising: a support tube distal portion attached to the actuation tube distal portion; a support tube proximal portion extending through the actuation tube proximal portion; a bendable section extending between the support tube distal portion and the support tube proximal portion; and at least one blade coupled to at least one arm of the bendable section; wherein axial movement of the actuation tube proximal portion and/or the support tube proximal portion relative to each other is configured to transition the bendable section between unbent and bent configurations thereof; and wherein the at least one blade is oriented at an angle relative to the actuation tube when the bendable section is in the bent configuration.

Example 2. The tissue cutting tool of any example herein, particularly example 1, wherein the at least one arm comprises a proximal arm and a distal arm.

Example 3. The tissue cutting tool of any example herein, particularly example 2, wherein the at least one blade comprises a blade attached to the proximal arm.

Example 4. The tissue cutting tool of any example herein, particularly example 3, further comprising a gathering arm attached, at a gathering arm distal portion thereof, to the distal arm.

Example 5. The tissue cutting tool of any example herein, particularly example 4, wherein the gathering arm comprises a gathering arm proximal portion extending proximally from the gathering arm distal portion.

Example 6. The tissue cutting tool of any example herein, particularly example 5, wherein the gathering arm proximal portion is not attached to the proximal arm.

Example 7. The tissue cutting tool of any example herein, particularly example 5 or 6, wherein the gathering arm proximal portion is oriented radially away from the blade in the bent configuration of the bendable section.

Example 8. The tissue cutting tool of any example herein, particularly example 2 or 3, wherein the at least one blade comprises a blade attached to the distal arm.

Example 9. The tissue cutting tool of claim 2, wherein the at least one blade comprises a blade attached to the distal arm, and wherein the tissue cutting tool further comprises a gathering arm attached, at a gathering arm proximal portion thereof, to the proximal arm.

Example 10. The tissue cutting tool of claim 9, wherein the gathering arm comprises a gathering arm distal portion extending distally from the gathering arm proximal portion.

Example 11. The tissue cutting tool of claim 10, wherein the gathering arm distal portion is not attached to the distal arm.

Example 12. The tissue cutting tool of claim 10 or 11, wherein the gathering arm distal portion is oriented radially away from the blade in the bent configuration of the bendable section.

Example 13. The tissue cutting tool of any one of claim 4-7 or 9-12, wherein the blade extends into a gathering arm slot of the gathering arm when the bendable section is in the unbent configuration.

Example 14. The tissue cutting tool of any one of claim 4-7 or 9-13, wherein gathering arm is atraumatic.

Example 15. The tissue cutting tool of any example herein, particularly any one of examples 1 to 14, wherein the actuation tube further comprises a cutout extending between the actuation tube distal portion and the actuation tube proximal portion.

Example 16. The tissue cutting tool of any example herein, particularly example 15, wherein the bendable section is aligned with the cutout.

Example 17. The tissue cutting tool of any example herein, particularly example 15 or 16, wherein the bendable section protrudes radially away from the cutout in the bent configuration.

Example 18. The tissue cutting tool of any example herein, particularly any one of examples 1 to 17, wherein the bendable section is configured to transition from the unbent configuration to the bent configuration when the support tube proximal portion is axially translated in a distal direction relative to the actuation tube proximal portion.

Example 19. The tissue cutting tool of any example herein, particularly any one of examples 1 to 18, wherein the bendable section is configured to transition from the unbent configuration to the bent configuration when the actuation tube proximal portion is axially translated in a proximal direction relative to the support tube proximal portion.

Example 20. The tissue cutting tool of any example herein, particularly any one of examples 1 to 19, wherein the bendable section is configured to transition from the unbent configuration to the bent configuration when the support tube proximal portion is axially translated in a proximal direction relative to the actuation tube proximal portion.

Example 21. The tissue cutting tool of any example herein, particularly any one of examples 1 to 20, wherein the bendable section is configured to transition from the unbent configuration to the bent configuration when the actuation tube proximal portion is axially translated in a distal direction relative to the support tube proximal portion.

Example 22. The tissue cutting tool of any example herein, particularly any one of examples 1 to 21, wherein the at least one blade comprises a sharp edge facing radially away from the at least one arm the blade is attached to.

Example 23. The tissue cutting tool of any example herein, particularly any one of examples 1 to 22, further comprising a dilator attached to the actuation tube, wherein the dilator comprises a dilator distal tapering surface that tapers towards a distal end of the dilator.

Example 24. The tissue cutting tool of any example herein, particularly example 23, wherein the dilator defines a dilator lumen which is open ended at the distal end of the dilator.

Example 25. The tissue cutting tool of any example herein, particularly example 23 or 24, wherein the dilator is attached to the actuation tube distal portion.

Example 26. The tissue cutting tool of any example herein, particularly any one of examples 1 to 22, wherein the actuation tube comprises an actuation tube distal edge configured to perforate a target tissue.

Example 27. The tissue cutting tool of any example herein, particularly example 26, wherein the actuation tube distal edge comprises an angled surface.

Example 28. The tissue cutting tool of any example herein, particularly example 26 or 27, wherein the actuation tube distal edge comprises a sharp actuation tube tip.

Example 29. The tissue cutting tool of any example herein, particularly any one of examples 1 to 22, further comprising a needle extending through the blade support tube.

Example 30. The tissue cutting tool of any example herein, particularly example 29, wherein the needle is axially movable relative to the blade support tube.

Example 31. The tissue cutting tool of any example herein, particularly example 29 or 30, wherein the needle comprises a needle head terminating at a needle tip.

Example 32. The tissue cutting tool of any example herein, particularly example 31, wherein the needle tip is configured to perforate a target tissue.

Example 33. The tissue cutting tool of any example herein, particularly any one of examples 29 to 32, wherein the blade support tube defines a support tube lumen sized to allow axial passage of the needle therethrough.

Example 34. The tissue cutting tool of any example herein, particularly any one of examples 29 to 33, wherein the needle defines a needle lumen sized to allow axial passage of a guidewire therethrough.

Example 35. The tissue cutting tool of any example herein, particularly any one of examples 1 to 28, wherein the blade support tube defines a support tube lumen sized to allow axial passage of a guidewire therethrough.

Example 36. The tissue cutting tool of any example herein, particularly any one of examples 1 to 22, further comprising a guidewire extending through a support tube lumen of the blade support tube.

Example 37. The tissue cutting tool of any example herein, particularly example 36, wherein the guidewire comprises a guidewire tip configured to penetrate through a target tissue.

Example 38. The tissue cutting tool of any example herein, particularly example 37, wherein the guidewire tip is a sharp tip.

Example 39. The tissue cutting tool of any example herein, particularly example 37 or 38, further comprising an RF energy source coupled to the guidewire and configured to provide RF energy to the guidewire tip.

Example 40. The tissue cutting tool of any example herein, particularly of any one of examples 26 or 32 or 37, wherein the target tissue is a host leaflet of a host valvular structure.

Example 41. The tissue cutting tool of any example herein, particularly example 20, wherein the host valvular structure is a native valvular structure of native heart valve.

Example 42. The tissue cutting tool of any example herein, particularly example 41, wherein the native heart valve is a native aortic valve.

Example 43. The tissue cutting tool of any example herein, particularly example 41, wherein the native heart valve is a native mitral valve.

Example 44. The tissue cutting tool of any example herein, particularly example 43, wherein the host leaflet is an anterior leaflet.

Example 45. The tissue cutting tool of any example herein, particularly example 40, wherein the host valvular structure is a valvular structure of previously implanted prosthetic valve that is implanted within a native heart valve.

Example 46. The tissue cutting tool of any example herein, particularly any one of examples 1 to 45, wherein the at least one arm comprises a retention portion to which the corresponding blade is coupled.

Example 47. The tissue cutting tool of any example herein, particularly example 46, wherein the retention portion comprises a slot defining two slot sidewalls facing each other, wherein at least one of the slot sidewalls comprises at least one protrusion extending therefrom toward the opposite sidewall.

Example 48. The tissue cutting tool of any example herein, particularly example 47, wherein each of the slot sidewalls comprises at least one of the at least one protrusion.

Example 49. The tissue cutting tool of any example herein, particularly example 47 or 48, wherein at least one of the slot sidewalls comprises a plurality of the at least one protrusion.

Example 50. The tissue cutting tool of any example herein, particularly any one of examples 46 to 49, wherein the at least one blade extends through the slot.

Example 52. The tissue cutting tool of any example herein, particularly any one of examples 46 to 50, wherein the at least one blade comprises at least one side opening through which the at least one protrusion extends.

Example 52. The tissue cutting tool of any example herein, particularly any one of examples 1 to 51, wherein the bendable section is made of a shape memory material.

Example 53. The tissue cutting tool of any example herein, particularly any one of examples 1 to 52, wherein the blade support tube is made of a shape memory material.

Example 54. The tissue cutting tool of any example herein, particularly example 52 or 53, wherein the shape memory material comprises Nitinol.

Example 55. The tissue cutting tool of any example herein, particularly any one of examples 1 to 54, wherein the support tube distal portion comprises at least one tab extending at a tab base thereof from the blade support tube, and terminating at a tab free end opposite to the tab base.

Example 56. The tissue cutting tool of any example herein, particularly example 55, wherein the at least one tab is radially biased radially outwards, such that the tab free end is radially offset relative to the tab base.

Example 57. The tissue cutting tool of any example herein, particularly example 56, wherein the actuation tube distal portion comprises at least one window defining a window distal edge and a window proximal edge, and wherein the tab free end of the at least one tab is engaged with at least one of the window distal edge or the window proximal edge.

Example 58. The tissue cutting tool of any example herein, particularly example 57, wherein the at least one windows comprises a plurality of windows, and wherein the at least one tab comprises a plurality of tabs engaged with the plurality of windows.

Example 59. The tissue cutting tool of any example herein, particularly example 58, wherein the tab free end of at least one of the plurality of tabs is engaged with the window distal edge of at least one of the plurality of windows.

Example 60. The tissue cutting tool of any example herein, particularly example 59, wherein engagement between the tab free edge and the window distal edge is configured to prevent axial movement in the distal direction of the support tube distal portion relative to the actuation tube distal portion.

Example 61. The tissue cutting tool of any example herein, particularly any one of examples 58 to 60, wherein the tab free end of at least one of the plurality of tabs is engaged with the window proximal edge of at least one of the plurality of windows.

Example 62. The tissue cutting tool of any example herein, particularly example 61, wherein engagement between the tab free edge and the window proximal edge is configured to prevent axial movement in the proximal direction of the support tube distal portion relative to the actuation tube distal portion.

Example 63. The tissue cutting tool of any example herein, particularly any one of examples 1 to 62, further comprising a cover shaft disposed around the actuation tube.

Example 64. The tissue cutting tool of any example herein, particularly example 63, wherein the cover shaft is axially movable with respect to the actuation tube.

Example 65. The tissue cutting tool of any example herein, particularly example 63 or 64, wherein the cover shaft is configured to conceal the bendable section in the unbent configuration, and expose the bendable section in the bent configuration.

Example 66. The tissue cutting tool of any example herein, particularly example 64, wherein a distal edge of the cover shaft is proximal to the bendable section in the bent configuration.

Example 67. The tissue cutting tool of any example herein, particularly any one of examples 63 to 66, further comprising a catheter disposed around the cover shaft.

Example 68. The tissue cutting tool of any example herein, particularly example 67, wherein catheter is axially movable with respect to the cover shaft.

Example 69. The tissue cutting tool of any example herein, particularly any one of examples 1 to 68, wherein the tissue cutting tool is sterilized.

Example 70. A method of forming an opening in a target tissue, the method comprising: advancing a tissue cutting tool to a target tissue, wherein the tissue cutting tool comprises: an actuation tube having an actuation tube distal portion and an actuation tube proximal portion, a blade support tube having a support tube proximal portion extending through the actuation tube proximal portion and a support tube distal portion attached to the actuation tube distal portion, and at least one blade coupled to at least one arm of a bendable section of the blade support tube, wherein the bendable section of the blade support tube extends between the support tube distal portion and the support tube proximal portion; transitioning the bendable section from an unbent configuration thereof to a bent configuration; and forming a tissue opening within the target tissue by axially moving the bendable section through the target tissue.

Example 71. The method of any example herein, particularly example 70, wherein the support tube distal portion comprises at least one tab extending at a tab base thereof from the blade support tube, and terminating at a tab free end opposite to the tab base.

Example 72. The method of any example herein, particularly example 71, wherein the at least one tab is radially biased radially outwards, such that the tab free end is radially offset relative to the tab base.

Example 73. The method of any example herein, particularly example 72, wherein the actuation tube distal portion comprises at least one window defining a window distal edge and a window proximal edge, and wherein the tab free end of the at least one tab is engaged with at least one of the window distal edge or the window proximal edge.

Example 74. The method of any example herein, particularly example 73, wherein the at least one windows comprises a plurality of windows, and wherein the at least one tab comprises a plurality of tabs engaged with the plurality of windows.

Example 75. The method of any example herein, particularly example 74, wherein the tab free end of at least one of the plurality of tabs is engaged with the window distal edge of at least one of the plurality of windows.

Example 76. The method of any example herein, particularly example 75, wherein engagement between the tab free edge and the window distal edge is configured to prevent axial movement in the distal direction of the support tube distal portion relative to the actuation tube distal portion.

Example 77. The method of any example herein, particularly example 75 or 76, wherein the tab free end of at least one of the plurality of tabs is engaged with the window proximal edge of at least one of the plurality of windows.

Example 78. The method of any example herein, particularly example 77, wherein engagement between the tab free edge and the window proximal edge is configured to prevent axial movement in the proximal direction of the support tube distal portion relative to the actuation tube distal portion.

Example 79. The method of any example herein, particularly any one of examples 70 to 78, wherein the support tube proximal portion is axially movable with respect to the actuation tube proximal portion.

Example 80. The method of any example herein, particularly any one of examples 70 to 79, wherein the actuation tube further comprises a cutout extending between the actuation tube distal portion and the actuation tube proximal portion.

Example 81. The method of any example herein, particularly example 80, wherein the bendable section is aligned with the cutout.

Example 82. The method of any example herein, particularly example 80 or 81, wherein the transitioning the bendable section to the bent configuration causes the bendable section to protrude radially away from the cutout.

Example 83. The method of any example herein, particularly any one of examples 70 to 82, wherein the at least one blade comprises a sharp edge facing radially away from the at least one arm the blade is attached to.

Example 84. The method of any example herein, particularly any one of examples 70 to 83, wherein the at least one arm comprises a retention portion to which the corresponding blade is coupled.

Example 85. The method of any example herein, particularly example 84, wherein the retention portion comprises a slot defining two slot sidewalls facing each other, wherein at least one of the slot sidewalls comprises at least one protrusion extending therefrom toward the opposite sidewall.

Example 86. The method of any example herein, particularly example 85, wherein each of the slot sidewalls comprises at least one of the at least one protrusion.

Example 87. The method of any example herein, particularly example 85 or 86, wherein at least one of the slot sidewalls comprises a plurality of the at least one protrusion.

Example 88. The method of any example herein, particularly any one of examples 84 to 87, wherein the at least one blade extends through the slot.

Example 89. The method of any example herein, particularly any one of examples 84 to 88, wherein the at least one blade comprises at least one side opening through which the at least one protrusion extends.

Example 90. The method of any example herein, particularly any one of examples 70 to 89, wherein the bendable section is made of a shape memory material.

Example 91. The method of any example herein, particularly any one of examples 70 to 90, wherein the blade support tube is made of a shape memory material.

Example 92. The method of any example herein, particularly any one of examples 90 or 91, wherein the shape memory material comprises Nitinol.

Example 93. The method of any example herein, particularly any one of examples 70 to 92, wherein the transitioning the bendable section to the bent configuration comprises axially translating the support tube proximal portion in a distal direction relative to the actuation tube proximal portion.

Example 94. The method of any example herein, particularly any one of examples 70 to 93, wherein the transitioning the bendable section to the bent configuration comprises axially translating the actuation tube proximal portion in a proximal direction relative to the support tube proximal portion.

Example 95. The method of any example herein, particularly any one of examples 70 to 93, further comprising, prior to the forming the tissue opening, forming a pilot puncture within the target tissue.

Example 96. The method of any example herein, particularly example 95, wherein the forming the tissue opening comprises passing the bendable section through the pilot puncture, such that the at least one blade cuts through the target tissue so as to expand the pilot puncture to form the tissue opening.

Example 97. The method of any example herein, particularly example 95 or 96, wherein the tissue cutting tool further comprises a needle extending through the blade support tube.

Example 98. The method of any example herein, particularly example 97, wherein the needle comprises a needle head terminating at a needle tip.

Example 99. The method of any example herein, particularly example 97 or 98, wherein the forming the pilot puncture comprises distally translating the needle through the target tissue.

Example 100. The method of any example herein, particularly of any one of examples 97 to 99, wherein the forming the pilot puncture comprises perforating the target tissue by a guidewire extending through the needle, followed by additionally piercing the target tissue by the needle, advanced over the guidewire to form the pilot puncture.

Example 101. The method of any example herein, particularly of any one of examples 97 to 100, wherein the tissue cutting tool further comprises a dilator attached to the actuation tube, wherein the dilator comprises a dilator distal tapering surface that tapers towards a distal end of the dilator.

Example 102. The method of any example herein, particularly of example 101, wherein the dilator is attached to the actuation tube distal portion.

Example 103. The method of any example herein, particularly of example 101 or 102, further comprising, subsequent to the forming the pilot puncture and prior to the forming the tissue opening, passing the dilator through the pilot puncture, thereby expanding the pilot puncture.

Example 104. The method of any example herein, particularly example 95 or 96, wherein the actuation tube comprises an actuation tube distal edge configured to cut through the target tissue.

Example 105. The method of any example herein, particularly example 104, wherein the actuation tube distal edge is distal to a distal edge of the support tube.

Example 106. The method of any example herein, particularly example 104 or 105, wherein the actuation tube distal edge comprises an angled surface.

Example 107. The method of any example herein, particularly any one of examples 104 to 106, wherein the forming the pilot puncture comprises distally translating the actuation tube distal edge through the target tissue.

Example 108. The method of any example herein, particularly any one of examples 104 to 107, wherein the forming the pilot puncture comprises perforating the target tissue by a guidewire extending through the actuation tube, followed by additionally piercing the target tissue by the actuation tube, advanced over the guidewire to form the pilot puncture.

Example 109. The method of any example herein, particularly example 95, wherein the forming the pilot puncture comprises perforating the target tissue by a guidewire extending through the actuation tube.

Example 110. The method of any example herein, particularly of any one of examples 100 or 108 or 109, wherein the forming the pilot puncture comprises applying RF energy to a tip of the guidewire.

Example 111. The method of any example herein, particularly any one of examples 70 to 108, wherein the advancing the tissue cutting tool to the target tissue keeping the bendable section concealed within a cover shaft lumen of a cover shaft disposed over the actuation tube.

Example 112. The method of any example herein, particularly example 111, further comprises, prior to the transitioning the bendable section to the bent configuration, axially retracting the cover shaft relative to the actuation tube, so as to exposed the bendable section.

Example 113. The method of any example herein, particularly example 111 or 112, wherein the advancing the tissue cutting tool to the target tissue comprises retaining the actuation tube and the blade support tube inside a catheter lumen of a catheter disposed around the cover shaft.

Example 114. The method of any example herein, particularly example 113, further comprises, prior to the transitioning the bendable section to the bent configuration, axially retracting the catheter so as to position a catheter distal edge thereof proximal to the bendable section.

Example 115. The method of any example herein, particularly any one of examples 70 to 114, further comprising, prior to the transitioning the bendable section to the bent configuration, axially transitioning the bendable section through the target tissue in the unbent configuration, so as to position the bendable section distal to the target tissue.

Example 116. The method of any example herein, particularly example 115, wherein the forming the tissue opening comprises axially pulling the bendable section in a proximal direction, through the target tissue.

Example 117. The method of any example herein, particularly example 116, wherein the at least one arm comprises a proximal arm, and wherein the at least one blade comprises a blade coupled to the proximal arm.

Example 118. The method of any example herein, particularly of example 117, wherein the at least one arm further comprises a distal arm, and wherein the tissue cutting tool further comprises a gathering arm attached, at a gathering arm distal portion thereof, to the distal arm.

Example 119. The method of any example herein, particularly of example 118, wherein the gathering arm comprises a gathering arm proximal portion extending proximally from the gathering arm distal portion.

Example 120. The method of any example herein, particularly of example 119, wherein the gathering arm proximal portion is not attached to the proximal arm.

Example 121. The method of any example herein, particularly of example 119 or 120, wherein the transitioning the bendable section to the bent configuration causes the gathering arm proximal portion to be oriented radially away from the blade.

Example 122. The method of any example herein, particularly any one of examples 119 to 121, wherein the axially pulling the bendable section comprises gathering a portion of the target tissue between the gathering arm proximal portion and the proximal arm of the bendable section.

Example 123. The method of any example herein, particularly any one of examples 70 to 114, wherein the transitioning the bendable section to the bent configuration is performed while the bendable section is positioned proximal to the target tissue.

Example 124. The method of any example herein, particularly example 123, wherein the forming the tissue opening comprises axially pushing the bendable section in a distal direction, through the target tissue.

Example 125. The method of any example herein, particularly example 124, wherein the at least one arm comprises a distal arm, and wherein the at least one blade comprises a blade coupled to the distal arm.

Example 126. The method of any example herein, particularly example 125, wherein the at least one arm further comprises a proximal arm, and wherein the tissue cutting tool further comprises a gathering arm attached, at a gathering arm proximal portion thereof, to the proximal arm.

Example 127. The method of any example herein, particularly example 126, wherein the gathering arm comprises a gathering arm distal portion extending distally from the gathering arm proximal portion.

Example 128. The method of any example herein, particularly example 127, wherein the gathering arm distal portion is not attached to the distal arm.

Example 129. The method of any example herein, particularly example 127 or 128, wherein the transitioning the bendable section to the bent configuration causes the gathering arm distal portion to be oriented radially away from the blade.

Example 130. The method of any example herein, particularly of any one of examples 127 to 129, wherein the axially pushing the bendable section comprises gathering a portion of the target tissue between the gathering arm distal portion and the distal arm of the bendable section.

Example 131. The method of any example herein, particularly of any one of examples 119-122 or 127-130, wherein the forming the tissue opening comprises forming a full cut extending to a free edge of the target tissue.

Example 132. The method of any example herein, particularly any one of examples 70-117 or 123-125, wherein the target tissue is the interatrial septum.

Example 133. The method of any example herein, particularly any one of examples 70 to 131, wherein the target tissue is a host leaflet of a host valvular structure, and wherein the tissue opening is a leaflet opening.

Example 134. The method of any example herein, particularly example 133, wherein the host valvular structure is a native valvular structure of native heart valve.

Example 135. The method of any example herein, particularly example 134, wherein the native heart valve is a native aortic valve.

Example 136. The method of any example herein, particularly example 134, wherein the native heart valve is a native mitral valve.

Example 137. The method of any example herein, particularly example 136, wherein the host leaflet is an anterior leaflet.

Example 138. The method of any example herein, particularly example 133, wherein the host valvular structure is a valvular structure of previously implanted prosthetic valve that is implanted within a native heart valve.

Example 139. The method of any example herein, particularly any one of examples 133 to 138, wherein the forming the leaflet opening comprises forming a first leaflet opening in a first host leaflet, and wherein, subsequent to forming the first leaflet opening, the method further comprises: retracting the tissue cutting tool from the first host leaflet; positioning the tissue cutting tool proximate to a second host leaflet; and forming a second leaflet opening within the second host leaflet by axially moving the bendable section, in the bent configuration, through the second host leaflet.

Example 140. The method of any example herein, particularly any one of examples 133 to 138, further comprising, subsequent to forming the leaflet opening: positioning a guest prosthetic valve in a radially compressed state within the host valvular structure; and radially expanding the guest prosthetic valve.

Example 141. The method of any example herein, particularly example 140, wherein the positioning the guest prosthetic valve within the host valvular structure comprises positioning the guest prosthetic valve within the leaflet opening.

Example 142. The method of any example herein, particularly example 140 or 141, wherein radially expanding the guest prosthetic valve comprises inflating a balloon over which the guest prosthetic valve is disposed.

Example 143. The method of any example herein, particularly example 140 or 141, wherein radially expanding the guest prosthetic valve comprises actuating a mechanical actuator of the guest prosthetic valve.

Example 144. The method of any example herein, particularly example 140 or 141, wherein the guest prosthetic valve is a self-expandable prosthetic valve, and wherein radially expanding the guest prosthetic valve comprises removing a restraint from around the guest prosthetic valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A tissue cutting tool, comprising:
an actuation tube comprising:
an actuation tube distal portion; and
an actuation tube proximal portion; and
a blade support tube comprising:
a support tube distal portion attached to the actuation tube distal portion;
a support tube proximal portion extending through the actuation tube proximal portion;
a bendable section extending between the support tube distal portion and the support tube proximal portion; and
at least one blade coupled to at least one arm of the bendable section,
wherein axial movement of the actuation tube proximal portion and/or the support tube proximal portion relative to each other is configured to transition the bendable section between unbent and bent configurations thereof; and
wherein the at least one blade is oriented at an angle relative to the actuation tube when the bendable section is in the bent configuration.

2. The tissue cutting tool of claim 1, wherein the at least one arm comprises a proximal arm and a distal arm.

3. The tissue cutting tool of claim 2, wherein the at least one blade comprises a blade attached to the proximal arm.

4. The tissue cutting tool of claim 3, further comprising a gathering arm attached, at a gathering arm distal portion thereof, to the distal arm.

5. The tissue cutting tool of claim 4, wherein the gathering arm comprises a gathering arm proximal portion extending proximally from the gathering arm distal portion.

6. The tissue cutting tool of claim 5, wherein the gathering arm proximal portion is oriented radially away from the blade in the bent configuration of the bendable section.

7. The tissue cutting tool of claim 2, wherein the at least one blade comprises a blade attached to the distal arm.

8. The tissue cutting tool of claim 1, wherein the actuation tube further comprises a cutout extending between the actuation tube distal portion and the actuation tube proximal portion.

9. The tissue cutting tool of claim 8, wherein the bendable section protrudes radially away from the cutout in the bent configuration.

10. The tissue cutting tool of claim 1, wherein the actuation tube comprises an actuation tube distal edge configured to perforate a target tissue.

11. The tissue cutting tool of claim 1, further comprising a needle extending through the blade support tube.

12. A method of forming an opening in a target tissue, the method comprising:
advancing a tissue cutting tool to a target tissue, wherein the tissue cutting tool comprises: an actuation tube having an actuation tube distal portion and an actuation tube proximal portion, a blade support tube having a support tube proximal portion extending through the actuation tube proximal portion and a support tube distal portion attached to the actuation tube distal portion, and at least one blade coupled to at least one arm of a bendable section of the blade support tube, wherein the bendable section of the blade support tube extends between the support tube distal portion and the support tube proximal portion;
transitioning the bendable section from an unbent configuration thereof to a bent configuration; and
forming a tissue opening within the target tissue by axially moving the bendable section through the target tissue.

13. The method of claim 12, wherein the actuation tube further comprises a cutout extending between the actuation tube distal portion and the actuation tube proximal portion.

14. The method of claim 13, wherein the transitioning the bendable section to the bent configuration causes the bendable section to protrude radially away from the cutout.

15. The method of claim 12, further comprising, prior to the forming the tissue opening, forming a pilot puncture within the target tissue.

16. The method of claim 15, wherein the forming the tissue opening comprises passing the bendable section through the pilot puncture, such that the at least one blade cuts through the target tissue so as to expand the pilot puncture to form the tissue opening.

17. The method of claim 15, wherein the tissue cutting tool further comprises a needle extending through the blade support tube.

18. The method of claim 17, wherein the forming the pilot puncture comprises distally translating the needle through the target tissue.

19. The method of claim 17, wherein the tissue cutting tool further comprises a dilator attached to the actuation tube, wherein the dilator comprises a dilator distal tapering surface that tapers towards a distal end of the dilator.

20. The method of claim 19, further comprising, subsequent to the forming the pilot puncture and prior to the forming the tissue opening, passing the dilator through the pilot puncture, thereby expanding the pilot puncture.

21. The method of claim 15, wherein the actuation tube comprises an actuation tube distal edge configured to cut through the target tissue.

22. The method of claim 21, wherein the forming the pilot puncture comprises distally translating the actuation tube distal edge through the target tissue.
